(19) United States
(12) Patent Application Publication
(10) Pub. No.: US 2004/0228831 A1
(43) Pub. Date: Nov. 18, 2004

(54) Polymeric conjugates for tissue activated drug delivery

(76) Inventors: Benjamin A. Belinka JR., Kendall Park, NJ (US); James J. Pachence, Hopewell, NJ (US); Jose G. Rosa, Lawrenceville, NJ (US); Paul M. Simon, Wilmington, DE (US)

Correspondence Address: PATENT ADMINSTRATOR, KATTEN MUCHIN ZAVIS ROSENMAN, 525 WEST MONROE STREET, SUITE 1600, CHICAGO, IL, 60661-3693, US

(21) Appl. No.: 10/437,983

(22) Filed: May 15, 2003

(51) Int. Cl. A61K 38/43

(52) U.S. Cl. 424/78.27; 424/94.1

(57) ABSTRACT

The present invention relates to a polymeric drug conjugate with one or more biologically active agents conjugated via an enzymatically cleavable linker to either a regular repeating linear unit comprising a water soluble polymer segment and a multifunctional chemical moiety, or a branched polymer comprising two or more water soluble polymer segments each bound to a common multifunctional chemical moiety, as well as to methods of making such conjugates. The present invention is also directed to pharmaceutical compositions comprising such conjugates and to the use of such conjugates to treat pathological conditions.

### 1. FIELD OF THE INVENTION

The present invention relates generally to polymeric drug conjugates composed of biologically active agents attached to regular repeating linear co-polymers or branched co-polymers by means of an enzymatically cleavable linker. In particular, the linker may contain one or more chemical bonds that are cleaved by enzymes, and, in some cases, may be further cleaved by changes in pH, ionic, or redox conditions. As such, the polymeric drug conjugates of the present invention can be specifically designed to provide for optimal enzymatic approach and cleavage of the linker by modifying the co-polymer. The present polymeric drug conjugates may be used to modify drug solubility, drug bioavailability, drug residence time, drug absorption characteristics, drug toxicity, and/or drug bioactivity.

### 2. BACKGROUND OF THE INVENTION

Novel formulation technologies have been developed to improve the delivery of many pharmaceutical agents, primarily to overcome issues of aqueous solubility, drug toxicity, bioavailability, and patient compliance (e.g., by increasing the time period between drug administrations). In many cases, clinical indications for important pharmaceutical agents, particularly anti-cancer drugs, are often dose-limited because of systemic toxicity. Novel drug delivery techniques can increase the therapeutic range of a compound by decreasing toxicity, thereby broadening the indications and clinical use of important drugs. For example, the toxicity of potentially important compounds can be significantly decreased by direct delivery of the active agent to specific tissues using implantable, bioresorbable polymers (Langer, 1990). However, implantable polymer technologies for drug delivery have inherent limitations such as the fact that drug release is often a function of hydrolytic degradation of the associated polymer or of simple diffusion of the drug from the polymer matrix. This means that the release characteristics are not a function of the disease being treated but are actually entirely independent of the disease.

Likewise, much of the previously described and known formulation technologies have limitations which will preclude their use with many types of drugs or many disease indications, and in general do not involve site-specific delivery technologies. For example, a widely utilized strategy for sustained delivery is to trap or encapsulate a drug into a lipid or polymer, limiting the availability of the drug to the biological system (Allen, et al., 1992; Thierry, et al., 1993; Tabata, 1993). In this case, the drug must either diffuse out of the capsule or polymer matrix, or the encapsulating agent must dissolve, disintegrate, or be absorbed before the drug can be released in a form which can be absorbed by the surrounding tissue. These techniques also rely on hydrolytic degradation for drug release. As such, polymer or lipid encapsulation systems do not provide for site-specific (targeted) release of a drug. In addition, polymer encapsulation systems are normally only suitable for water-soluble drugs, while liposomal formulations are restricted to those lipid soluble drugs which will partition in the liposomal bilayers without disruption of the bilayer integrity.

Macromolecules in the form of synthetic, natural, or semi-synthetic (chemically modified natural macromolecules) polymers have been utilized as carriers for a variety of pharmaceutical agents (e.g., Pachence and Kohn, 1998). Various chemical spacer groups have been previously used to covalently couple active agents to a polymer to create a conjugate capable of controlled or sustained release of a drug within the body. These spacer groups provide biodegradable bonds that permit controlled drug release. The size and nature of the spacer groups, and the charge and structure of the polymer are important characteristics to consider in the design of a drug with controlled or sustained release characteristics. As a result, the release of a drug often depends on hydrolytic cleavage of the bond between the polymer and active agent. However, while such a method does provide sustained release, it does not necessarily target the drug to a specific tissue.

Although polymeric drug conjugates can in general provide a method for controlled drug release or directed drug distribution in the body (and thereby improve the drug therapeutic index), the successful application of polymeric drug delivery systems depends to a great extent on: (1) the ability to reproducibly prepare well-defined polymer/drug conjugates; (2) providing an adequate payload (i.e., the ratio of drug molecular weight (MW) to polymer MW must be maximized); and (3) the choice of a linking group to attach the drug to the polymer. Particularly with natural and semi-synthetic polymers, covalent attachment of drugs to a polymer in general will lead to a random distribution along the polymer backbone. The spacing between each attached active agent is therefore random, and in general not controllable.

Various proteolytic enzymes are produced in greater quantity by cells near or at the site of disease, or at the site of infection by microbes or host cells. For example, matrix metalloproteinases (MMPs) are a major family of enzymes which regulate extracellular matrix composition and modulate the interaction between cells and ECM (Massova, et al., 1998). In addition to the normal role of MMPs in healing and metabolism, this enzyme family is also implicated in various pathological processes, including chronic inflammation, arthritis, and cancer. In particular, MMPs have been found to be active during tumor growth and to be necessary for metastasis (Chambers and Matrisian, 1997).

Furthermore, numerous enzymes are produced by pathogens at the site of an infection or by host cells (such as leukocytes) that are involved in combating infection. Thrombin-like alanine aminopeptidase and elastase-like enzymatic activity are known to be common in bacterial infections (Finlay and Cossart, 1997), and the amino acid cleavage sequences of such enzymes are well-documented.

Knowledge of enzyme production or up-regulation due to pathological conditions has been previously used as a strategy for drug delivery. For example, it has been shown that amino acid sequences known to be cleaved by specific enzymes present at the site of an infection can be coupled to an antibiotic, which can subsequently be incorporated into a polyvinyl alcohol hydrogel wound dressing (Suzuki, et al., 1998). Antibiotics such as gentamicin can thus be selectively released into infected wound exudate. Likewise, enzymes which are produced by cancer cells, such as serine protease prostate-specific antigen, can be used to activate prodrugs (Denmeade, et al., 1998).

More recently, a number of novel polymer/drug conjugates have been described in the literature. Kopecek, et al. (U.S. Pat. Nos. 5,037,883 and 5,258,453) describe the use of polymeric carriers attached to drugs with a linking chain that is cleaved by intracellular enzymes. This method, however, is limited by the necessity that the conjugate be taken into the cell before enzymatic cleavage can occur. Particularly, Kopecek, et al., describe a polymer with a targeting moiety wherein degradation of the drug-carrier linkage occurs via intracellular lysosomal hydrolysis. This technology further relies on chemically linking drugs to pre-formed polymers. This method limits the amount of drug bound to the polymer (typically less than 50% of the potential linking sites), and the resulting conjugate does not have a regularly repeating drug unit.

In a similar fashion, others have identified methods for releasing drugs coupled to polymers by relying on biodegradation of the bond between the drug and the polymer. For example, Thorpe (U.S. Pat. No. 5,474,765) describes a two component system consisting of a polyanionic polymer and a steroid linked via a hydrolyzing chemical bond. The tissue-targeting component of Thorpe is the endothelial cell-binding portion of heparin and similar polymers. Thorpe uses sulfated polyanionic polysaccharides (such as heparin) as the primary polymer constituent, and contemplates the use of synthetic organic sulfated polymers (such as polystyrene sulfonate, sulfated polyvinyl alcohol, or polyethylene sulfonate). According to Thorpe, the active agents are randomly conjugated to pre-formed polymers. However, these polymers are not water soluble, nor are they taught to extend drug residence time. In addition, the biologically releasable bonds linking the active agent to the polymer in Thorpe, are generally hydrolyzable and are not disease specific. As a result, Thorpe does not describe drug releasing conditions which would lead to tissue-localized high concentrations of active agent.

Other groups have described alternating PEG co-polymers that result in water-soluble polymers for drug delivery. For example, Zalipsky, et al. (U.S. Pat. Nos. 5,219,564 and 5,455,027) describe a linear pre-formed polymer of PEG and the amino acid lysine to result in functional pendant groups (such as the terminal carboxyl group of lysine) at regular intervals. However, these methods do not provide drug attachment along the polymer backbone at regular intervals. Likewise, the concept of enzymatic cleavage which would provide for site-directed drug delivery is not disclosed.

Polymers, such as those described by Zalipsky, have been used by others to provide methods of drug release. For example, Huang, et al. (Bioconjugate Chem. 9:612-617, 1998) conjugates cysteine-containing peptides to a PEG-lysine co-polymer (modified to provide regularly spaced thiol groups) using a disulfide linkage. Poiani, et al. (Bioconjugate Chem. 5:621-630, 1994; U.S. Pat. Nos. 5,372,807, 5,660,822, and 5,720,950) utilizes these same PEG-lysine with the anti-fibrotic compound cis-hydroxyproline. As with Zalipsky, neither Huang nor Poiani describe methods of providing drug attachment along the polymer backbone at regular intervals, nor is the concept of enzymatically cleavable linking groups described.

Other technologies provide methods for attaching polymers to active agents (particularly protein-based pharmaceutical compounds) as a means for creating a prodrug. For example, Greenwald, et al. (U.S. Pat. No. 5,840,900) describe the covalent attachment of polyethylene glycol (PEG) to active agents to create prodrugs. Greenwald, however, describes a compound that relies on hydrolytic cleavage of large molecular weight PEG's (at least 20,000) to reconstitute the active agents. Small organic drugs would be inappropriate according to the method of Greedwald, as the ratio of PEG to drug would be too high. In addition, Greenwald does not consider the use of linking groups which are cleavable at the disease site, and the resulting conjugate is not a regular polymer repeating structure.

A number of others (e.g. U.S. Pat. Nos. 4,753,984, 5,474,765, 5,618,528, 5,738,864, 5,853,713) describe technologies which link active agents to pre-formed polymers, but are limited to a single class of active agents and do not describe methods for creating a regularly repeating polymer construct.

### 3. SUMMARY OF THE INVENTION

The present invention relates generally to polymeric drug conjugates composed of biologically active agents attached to regular repeating linear co-polymers or branched co-polymers by means of an enzymatically cleavable linker. More specifically, the present invention relates to a polymeric drug conjugate comprising one or more biologically active agents conjugated via an enzymatically cleavable linker to either (i) a regular repeating linear unit comprising a water soluble polymer segment and a multifunctional chemical moiety, or (ii) a branched polymer comprising two or more water soluble polymer segments each bound to a common multifunctional chemical moiety. In particular, the linker contains one or more chemical bonds that may be cleaved by enzymes and, in some cases, additionally by changes in pH, ionic, or redox conditions, which are present in high concentration near, in, and/or on the surface of diseased tissues. The conjugates of the present invention can be designed to provide for optimal enzymatic approach to and cleavage of the linker by modifying the water soluble polymer segments of the linear co-polymer and/or the multifunctional chemical moieties of the branched co-polymer. The polymeric drug conjugate of the present invention may be used to modify drug solubility, drug bioavailability, drug residence time, drug absorption characteristics, and/or drug bioactivity.

General structures of preferred embodiments of the polymeric drug conjugates according to the present invention are shown below.

Polymeric Drug Conjugate Formulas

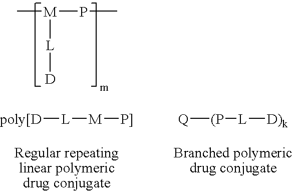

The co-polymer backbones of the conjugate of the present invention are composed of water soluble polymer segments attached to multifunctional chemical moieties to form either regular linear repeating or branched scaffolds. These scaffolds are designed to provide a series of evenly spaced chemical functionalities for the attachment of biologically active moieties via an enzymatically cleavable linking group. The composition of the co-polymer can be modified to allow for optimal enzymatic approach to the enzymatically cleavable linkers by the modification of the size or chemical structure of the individual polymer segments and/or the multifunctional chemical moieties.

Construct Formula poly[D-L-M-P]

The polymer construct poly[D-L-M-P] consists of a multifunctional chemical moiety, M, that is used to join water soluble polymer segments, P, to form a regular repeating linear co-polymer backbone and additionally to provide the chemical substituents for the attachment of a biologically active agent, D, via an enzymatically cleavable linker, L. The number of M-P repeats of the regular repeating linear co-polymer is designated by m.

The water soluble polymeric drug conjugate can be designed to increase the water solubility of D and be formulated to be administered through injection, oral, topical, inhalation delivery, subcutaneous deposition (implant), deposition using minimally invasive surgical procedures such as laproscopy, or other physical delivery methods.

The pharmaceutical agent released from the co-polymer/linking agent conjugate by enzymatic activity provides a high concentration of reconstituted pharmaceutical activity at a targeted tissue site. In general, the metabolically sensitive linking group, L, is designed to be cleaved by enzymes that are present in high concentration (higher than non-pathological levels) at or near the targeted disease site.

Construct Formula Q(-P-L-D)ₖ

The branched polymeric drug conjugate of the formula Q(-P-L-D)ₖ consists of a common multifunctional chemical moiety, Q, that is used to attach k number of water soluble polymer segments, P, attached to a biologically active agent, D, via an enzymatically cleavable linker, L. The water soluble polymeric drug conjugate can be designed to increase the water solubility of D and can be formulated to be administered through injection, oral, topical, inhalation delivery, subcutaneous deposition, deposition using minimally invasive surgical procedures (e.g. laproscopy), or other physical delivery methods well-known in the art.

The pharmaceutical agent released from the polymeric conjugate by enzymatic activity provides reconstituted pharmaceutical activity in a high concentration at the local, targeted tissue site. In general, the metabolically sensitive linking group, L, is designed to be cleaved by enzymes that are present at high concentration (higher than non-pathological levels) at or near the site of disease.

The present invention provides novel compositions for creating tissue-targeting formulations consisting of pharmaceutical agents conjugated to water soluble polymers. The present invention provides a method of targeting drug release by using chemical linking groups between the polymer and the pharmaceutical agent that would be specifically cleaved at the site of disease. In addition, the pharmaceutical agents according to the present invention are spaced along the water soluble polymer backbone at regular intervals, wherein the interval between each active agent is controlled by synthetic methods.

As a result, a unique aspect of the conjugate of the present invention is a construct which consists of a drug and linker which repeats on a water soluble polymer backbone. The spacing between attachment of the drug/linker group complex can be controlled by the synthetic methods presented herein. One preferred method of forming the conjugate of the present invention, is that the drug, linker, and monomer be conjugated first, and then the resulting product is coupled to a water soluble polymer which then forms the polymer conjugate. This provides for a high degree of drug-linker substitution on the polymer construct (typically greater than 90%), providing a regular repeating unit of the drug/linker along the polymer backbone.

The present invention also requires that the linking group be cleavable by enzymatic activity, such as by enzymes which may be present in high concentrations near, in and/or on the surface of diseased tissue. This unique aspect provides a mechanism for obtaining target site-directed drug delivery.

### 4. BRIEF DESCRIPTION OF THE DRAWINGS

FIG. 1A depicts results of treatment of mice bearing murine melanoma B16-F10 with a polymeric prodrug conjugate in which the drug moiety is attached to the PEG backbone via a cathepsin B-cleavable linker peptide.

FIG. 1B depicts results of treatment of mice bearing the murine colon cancer MC-38 with a polymeric prodrug 5-fluorouracil (5FU)-containing conjugate in which the drug moiety is attached via a cathepsin B-cleavable peptide linking group.

FIG. 1C depicts results of treatment of C57B1/6 mice bearing s.c. B16-F10 murine melanoma with a plasmin labile construct, 26, bearing an aspartic acid-platinum-diaminocyclohexane (DACH) chelate.

FIG. 1D depicts results of treatment of mice with a polymeric conjugate, 31, (VEO-066) bearing the uPA-cleavable peptide -Pro-Gly-Arg-, and Pt chelated through an aspartic acid residue and DACH.

FIG. 1E depicts results of treatment of athymic (nu/nu) mice bearing s.c. human colon cancer tumor HT-29 with a polymeric Dox-containing conjugate construct, 6, (VEO-0003) in which the drug moiety is attached via a cathepsin-B-cleavable (-Gly-Phe-Leu-Gly-) moiety.

### 5. DETAILED DESCRIPTION OF THE INVENTION

The present invention describes polymeric drug conjugates formed by covalently attaching a biologically active agent to a co-polymeric backbone via an enzymatically cleavable linker. The linker consists of chemical chains with one or more bonds that are susceptible to physiological cleavage, preferably enzymatic cleavage. The conjugates are administered to a patient, wherein the biologically active agent is released from the polymer backbone by a physiological process, and the biological agent's activity is reconstituted. The pharmaceutical agent released from the polymer/linking agent conjugate by metabolic activity provides reconstituted pharmaceutical activity in high concentrations at a specific tissue location.

The general structure of preferred constructs according to the present invention are shown below.

5.1 Polymeric Drug Conjugate Formulas

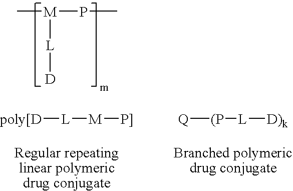

Construct Formula poly[D-L-M-P]

The polymer construct of formula poly[D-L-M-P] consists of a multifunctional chemical moiety, M, that is composed of organic compounds, amino acids, or a combination of both, which contains chemical functionalities that can be used to form covalent bonds with polymer segments, P, and the linking group, L. L is a linking group that can consist of, either independently or in combination, amino acids, sugars, nucleic acids, or other organic compounds which possess one or more chemical bonds that are enzymatically cleavable. D is a biologically active agent with a chemical substituent that can form a covalent bond to the linking group, L. P is a water soluble polymer or co-polymer with at least two functionalities that can form covalent chemical bonds to substituents on the monomer, M. m is the number of polymer repeats, typically ranging from about 2 to about 25, preferably from about 5 to about 12.

Construct Formula Q(-P-L-D)ₖ

The polymer construct of formula Q(-P-L-D)ₖ consists of a common multifunctional chemical moiety, Q, that is used to attach k number of water soluble polymer segments, P, and an enzymatically cleavable linker, L, that connects P to D, a biologically active agent, wherein k is an integer greater than 2, preferably an integer from about 2 to about 100, most preferably an integer from about 4 to about 8.

The water soluble polymer conjugate can be designed to increase the water solubility of D and can be formulated to be administered through injection, oral, topical, inhalation delivery, subcutaneous deposition, deposition using minimally invasive surgical procedures (such as laproscopy), or other well known physical delivery methods.

Another aspect of the invention is that the polymer construct Q(-P-L-D)ₖ allows for multiple equally spaced drug-linker substituents on each common multifunctional chemical moiety, Q. The structure, chemical composition, or size of the polymer segment, P, can be easily changed to allow for facile approach of the enzyme to the enzymatically cleavable linker, L, and optimize the biological utility of the construct product.

5.2 Assembly of the Polymeric Drug Conjugates

The polymeric drug conjugate poly[D-L-M-P] of the invention can be assembled using four separate units: the multifunctional chemical moiety, M, an enzymatically cleavable linker, L, a biologically active agent, D, and a water soluble polymer segment, P. These individual units are initially substituted with one or more reactive functional groups that are used to form stable chemical bonds with the other units of the construct.

Construct Q(-P-L-D)ₖ of the invention can also be assembled using four units: the common multifunctional chemical moiety, Q, an enzymatically cleavable linker, L, a biologically active agent, D, and a water soluble polymer segment, P. These individual units are initially substituted with one or more reactive functional groups that are used to form stable bonds with the other units of the construct.

5.3 Description of the Units of the Polymeric Drug Conjugates

The Multifunctional Chemical Moiety, M

The multifunctional chemical moiety, M, is designed to covalently join the water soluble polymer segments, P, and also to bind the linker, L. The multifunctional chemical moiety may be derived from a chemical compound comprising up to 50 carbon atoms and possessing multiple reactive chemical functionalities. M is preferably designed and synthesized to provide the structure and chemical functionalities illustrated in General Formula 1 below.

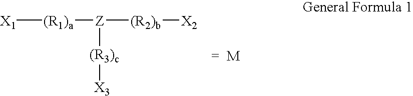

X₁, X₂, and X₃ are chemical substituents that can be used to form covalent bond with the polymer segments, P, or the enzymatically cleavable linker, L. X₁, X₂, and X₃ are independently selected from or derived from, hydroxyl, amino, thiol, alkyl disulfide, aryl disulfide, isothiocyanate, aldehyde, ketone, carboxylic acid, sulfonic acid, phosphoric acid, alkyl carbonate, aryl carbonate, succinimidyl carbonate, halide, or thioester functions (possibly substituted with appropriate protecting groups that can be removed before further chemical reaction) and the like.

R₁, R₂, and R₃ act as spacers that initially separate the reactive functional groups to provide an optimal chemical and stearic environment for the assembly of the polymeric drug conjugate, and ultimately separate the polymer segments, P, and linker, L, to allow for optimal biological activity of the construct. R₁, R₂, and R₃ are independently selected from the group consisting of saturated and unsaturated, straight and branched alkyl, aryl, alkylaryl, heteroalkyl, heteroaryl, or heteroalkyaryl chains which may contain up to 20 carbon atoms.

a, b, and c are integers, which, independently from one another, have a value of 0 to about 2.

Z is C, CH, N, P, PO, aryl, or heteroaryl.

The Linker, L

The enzymatically cleavable linker L, is illustrated in General Formula 2, where X₄ and X₅ are chemical substituents that can be used to form covalent bonds with the multifunctional chemical moiety, M, and the biologically active agent, D. X₄ and X_{5 are} independently selected from or derived from, hydroxyl, amino, thiol, alkyl disulfide, aryl disulfide, isothiocyanate, aldehyde, ketone, carboxylic acid, sulfonic acid, phosphoric acid, alkyl carbonate, aryl carbonate, succinimidyl carbonate, halide, or thioester functions (possibly substituted with appropriate protecting groups that can be removed before further chemical reaction) and the like.

General Formula 2

X₄−(R₄)_{d}−(L₁−Lₙ)−(R₅)ₑ−X₅=L

R₄ and R₅ act as spacers that initially separate the reactive functional groups to provide an optimal chemical and stearic environment for the assembly of the polymeric drug conjugate, and ultimately separate the linker, L, from the biologically active agent, D, and the multifunctional chemical moiety, M, to allow for optimal biological activity of the construct. R₄ and R₅ are independently selected from the group consisting of saturated and unsaturated, straight and branched alkyl, aryl, alkylaryl, heteroalkyl, heteroaryl, or heteroalkyaryl chains which may contain up to 20 carbon atoms.

d and e are integers, which, independently from one another, have a value of 0 to about 2.

(L₁-Lₙ) is a chain consisting of, either independently or in combination, amino acids, sugars, nucleic acids, or other organic compounds which possess at least one enzymatically cleavable bond.

The Biologically Active Agent, D

The biologically active agent, D, consists of any biologically useful agent, analog, or metabolite, or mixtures thereof, which possess (or can be modified to possess) at least one chemical functionality (for example, a hydroxyl, amino, thiol, alkyl or aryl disulfide, isothiocyanate, aldehyde, ketone, isothiocyanate, carboxylic acid, sulfonic acid, phosphoric acid, alkyl, aryl, or succinimidyl carbonate, halide, or thioester) for covalent attachment to the linker, L.

Biologically active agents that may be delivered by the conjugates of the present invention include, but are not limited to, analgesics, anesthetics, antifungals, antibiotics, antiinflammatories, anthelmintics, antidotes, antiemetics, antihistamines, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, sedatives, sulfonamides, stimulants, sympathomimetics, tranquilizers, urinary antiinfectives, vasoconstrictors, vasodilators, vitamins, xanthine derivatives, and the like. The biologically active agents may also be other small organic molecules, naturally isolated entities or their analogs, organometallic agents, chelated metals or metal salts, peptide based drugs, or peptidic or non-peptidic receptor targeting or binding agents.

The Water Soluble Polymer Segment, P

The water soluble polymer segment, P, is preferably a relatively short, water soluble polymeric system (for example, with an average MW of about 400 to about 25,000) which contains at least two chemical functionalities (for example, including but not limited to, hydroxyl, amino, thiol, alkyl or aryl disulfide, isothiocyanate, aldehyde, ketone, carboxylic acid, sulfonic acid, phosphoric acid, alkyl or aryl or succinimidyl carbonate, halide, or thioester) that can be used for covalent attachment to the multifunctional chemical moiety, M. More specifically, P may be poly(ethylene glycol), poly(vinyl alcohol), poly(2-hydroxyethyl methacrylate), poly(acrylic acid), poly(methacrylic acid), poly(maleic acid), or a co-polymer consisting of mixtures thereof or other polymeric entities possibly substituted with organic functional groups.

The Common Multifunctional Chemical Moiety, Q

The common multifunctional chemical moiety, Q, is designed to couple k number of soluble polymer segments, P. Q is preferably designed and synthesized to provide the structure and chemical functionalities illustrated in General Formula 3, shown below.

General Formula 3

J(-X₆)ₖ

X₆ is a chemical substituent that can be used to form covalent bonds with the polymer segments, P. X₆ can be selected from or derived from the group consisting of hydroxyl, amino, thiol, alkyl or aryl disulfide, isothiocyanate, aldehyde, ketone, carboxylic acid, sulfonic acid, phosphoric acid, alkyl or aryl or succinimidyl carbonate, halide, or thioester functions (possibly substituted with appropriate protecting groups that can be removed before further chemical reaction).

J acts as a spacer that initially separates the reactive functional groups to provide an optimal chemical and stearic environment for the assembly of the final polymeric drug conjugate, and ultimately separates the polymer segments, P, to allow for optimal biological activity of the construct. J may be a saturated and unsaturated, straight and branched alkyl, aryl, alkylaryl, heteroalkyl, heteroaryl, or heteroalkyaryl chain which may contain up to 20 carbon atoms.

5.4 Preparation of the Units of the Polymeric Drug Conjugates

Preparation of the Enzymatically Cleavable Linker, L

The linker, L, is assembled using standard synthesis methodologies and is designed to connect individual biologically active agents, D, to the polymeric scaffolds of the polymeric drug conjugates. L is also engineered to possess one or more enzymatically cleavable bonds, the breaking of which allows for the release of the biologically active agent or its analog from the polymeric constructs. The linker, L, may also include spacer groups, R₄ & R₅, that contain one or more hydrolytically, oxidatively, or photolytically cleavable chemical bonds. Since L contains two active functional groups, X₄ & X₅, one or more of these functions can be chemically protected during the assembly of the construct and then de-protected as required. A detailed list of chemical protecting groups and de-protection conditions can be found in Greene, et al., “Protective Groups in Organic Synthesis,” John Wiley & Sons, New York, 1981, herein expressly incorporated by reference in its entirety.

The enzymatically cleavable bond of the linker may be spaced from the co-polymeric backbone and biologically active agent via spacer groups to allow for enhanced exposure of the linking group to enzymes or to provide an optimal chemical environment for cleavage.

The assembly of L, which is selected from the group consisting of amino

acids, sugars, nucleic acids, or other organic compounds joined by saturated and unsaturated, straight and branched chain alkyl, aryl, or alkylaryl, heteroalkyl, heteroaryl, or heteroalkyaryl groups which may contain up to 20 carbon atoms, can be accomplished by using reagents and techniques well known to one of ordinary skill in the art.

In a prefered embodiment, the enzymatically cleavable linker, L, is derived from the peptide H-Gly-Phe-Gly-Gly(5-fluorouracil-1-yl)-OEt which can be assembled using known amino acid coupling techniques (see Kopecek, et al. Bioconjugate Chemistry, 1995, 6, 483 and Lloyd-Williams, et al. “Chemical Approaches to the Synthesis of Peptides and Proteins,” CRC Press, New York, 1997, herein expressly incoporated by reference in its entirety).

In another preferred embodiment, the cleavable linker is derived from the tetrapeptide, H-Gly-Phe-Leu-Gly-OH.

In another preferred embodiment, the cleavable linker is derived from the peptide, H-Ser-Ser-Ser-Pro-Leu-Ala-Nva-Gly-Ala-OH.

In another preferred embodiment, the cleavable linker is derived from the peptide, H-Ser-Ser-Ser-Gly-Pro-Asn-Gly-Ile-Ala-Gly-Asn-Asp-OH.

Preparation of the Multifunctional Chemical Moiety, M

The multifunctional chemical moiety, M, is prepared using reagents and techniques well known to one skilled in the art. Since M contains three active functional groups, X₁, X₂, & X₃, one or more of these functions can be chemically protected during synthesis and then de-protected as required. For a description of the reactions which can be used to attach one portion of the construct to another, see March, J., Advanced Organic Chemistry, 4th Edition, John Wiley & Sons, New York, 1992, expressly herein incorporated by reference. For a description of the reactions that can be used to prepare reactive functionalities on organic, peptide, and nucleotide moieties, see Larock, R. C., Comprehensive Transformations, VCH, New York, 1989; Bodansky, M., Principles of Peptide Synthesis, Springer-Verlag, New York, 1984; and Mizuno, Y., The Organic Chemistry of Nucleic Acids, Elsevier, New York, 1986, each of which is expressly herein incorporated by reference.

The structures of six preferred multifunctional chemical moieties, M, or their precursors are shown below in Diagram 1.

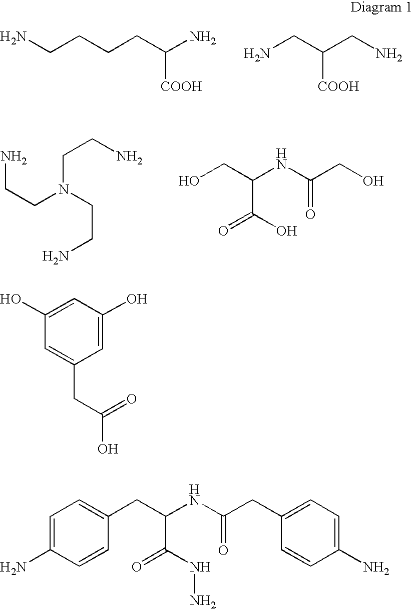

Preparation of the Common Multifunctional Chemical Moieties, Q

The multifunctional chemical moieties, Q, used to prepare the branched polymeric drug conjugates of the present invention are prepared using reagents and techniques well known to one of skill in the art. Since Q contains multiple reactive functions X₆ that are designed to react with the polymer segments, P, alone, no protecting groups are usually required. In some cases, however, when more than one type of biologically active agent, D, enzymatically cleavable linker, L, or water soluble polymer segment, P, are desired in a single polymeric drug conjugate, some of the reactive functional groups may be protected during certain synthetic steps and then de-protected when required. For a description of the reactions which can be used to attach one portion of the construct to another see March, J., Advanced Organic Chemistry, 4th Edition, John Wiley & Sons, New York, 1992, herein incorporated by reference in its entirety. For a description of the reactions that can be used to prepare reactive functionalities on organic, peptide, and nucleotide moieties, see Larock, R. C.: Comprehensive Transformations, VCH, New York, 1989, Bodansky, M.; Principles of Peptide Synthesis, Springer-Verlag, New York, 1984, and Mizuno, Y; The Organic Chemistry of Nucleic Acids, Elsevier, New York, 1986, each of which are herein incorporated by reference in their entirety.

The structures of two preferred multifunctional monomer, Q, are illustrated below in Diagram 2.

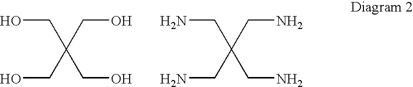

5.5 Polymeric Drug Conjugate Assembly Pathways

There are several synthetic pathways that can be used to assemble the polymeric drug conjugates described herein.

Construct, poly[D-L-M-P]

5.5.1 Assembly Method I

The method of assembly, illustrated below in Reaction Scheme 1, requires an initial covalent coupling of the linker, L, to the biologically active agent, D, producing a construct, D-L. The D-L construct is then reacted with the multifunctional chemical moiety, M, producing the D-L-M system.

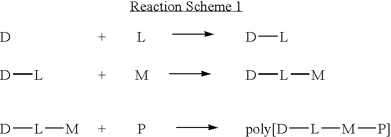

The D-L-M system is then covalently coupled to the appropriate water soluble polymeric segment, P, to give the desired regular repeating linear polymeric drug conjugate poly[D-L-M-P].

5.5.2 Assembly Method II

Alternatively, the linker, L, can first be coupled to M, to form L-M as shown below in Reaction Scheme 2.

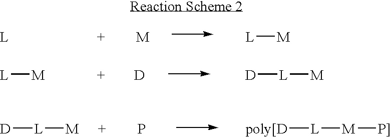

The second reaction of this pathway requires the chemical coupling of L-M to the biologically active agent, D, yielding the D-L-M construct which is then attached to the appropriate polymeric system, P, to form the construct poly[D-L-M-P] of the present invention.

5.5.3 Assembly Method III

Alternatively, M can be initially coupled to the linker, L, as shown in Reaction Scheme 3 below, to prepare conjugate L-M. Conjugate L-M is then reacted with P and then D to prepare the construct of the present invention.

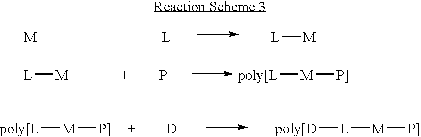

See General Formula 4, below, for a description of the structural abbreviations.

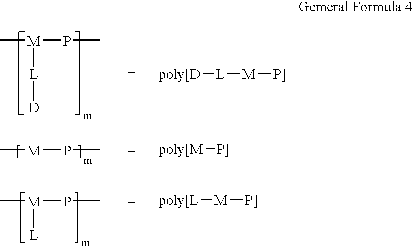

5.5.4 Assembly Method IV

Alternatively, M can be covalently attached to the polymer, P, to yield the construct poly[M-P] (see Reaction Scheme 4 below). The co-polymer conjugate is then reacted with the linker, L, to yield poly[L-M-P] and is then coupled to the biologically active agent, D, to yield the product poly[D-L-M-P].

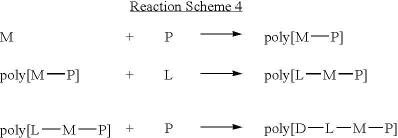

5.5.5 Assembly Method V

Alternatively, M can be reacted with P to form polymer conjugate, poly[M-P], as shown in Reaction Scheme 5, below.

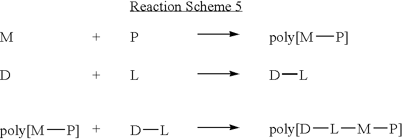

The D-L construct can then be synthesized independently and attached to poly[M-P], to yield the poly[D-L-M-P] construct of the present invention.

Construct, Q(-P-L-D)ₖ

5.5.6 Assembly Method VI

The assembly of the branched polymeric drug conjugate Q(-P-L-D)ₖ is illustrated in Reaction Scheme 6, below. An initial covalent coupling of the linker, L, to the biologically active agent, D, and the conjugation of the common multifunctional chemical moiety to two or more water soluble polymer segments is followed by the attachment of both resulting conjugates to produce the invented construct.

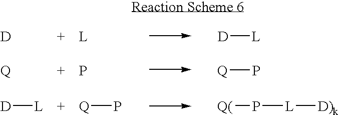

5.5.7 Assembly Method VII

Alternatively, Q can first be coupled to two or more polymer segments, P, to form Q(-P)ₖ. (See Reaction Scheme 7, below.)

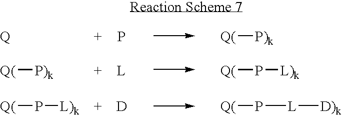

The second reaction of this pathway requires the chemical coupling of Q-(P)ₖ to the linker L, yielding the Q-(P-L)ₖ macromer which is then attached to the appropriate biologically active agent, D, to form the construct Q(-P-L-D)ₖ

5.5.8 Assembly Method VIII

Alternatively, the biologically active agent, D, can be initially coupled to the linker, L, (see Reaction Scheme 8, below) to prepare conjugate L-D. L-D is then reacted with P to produce the macromer P-L-D which is then attached to the common multifunctional moiety, Q, to prepare the construct of the present invention.

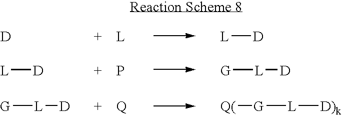

5.6 Chemical Methods for the Assembly of the Polymeric Drug Conjugates, poly[D-L-M-P] and Q(-P-L-D)ₖ

The constructs of the present invention are assembled by the covalent coupling of structural portions, D, L, P and M or D, L, P and Q. These units are attached by means of the substituents X₁-X₆ which are preferably chosen to allow for the formation of stable covalent bonds between the various units of the polymeric drug conjugate.

Attachment of the Pharmaceutically Active Agent, D, to the Linker, L

The pharmaceutical agents, analogs, or metabolites, D, possess or can be modified to possess, reactive substituents for the formation of covalent bonds with linker, L. Alternatively, a spacer group can be attached to D to allow for attachment to L. As discussed above, theses substituents are independently selected from or derived from hydroxyl, amino, thiol, alkyl or aryl disulfide, isothiocyanate, aldehyde, ketone, carboxylic acid, sulfonic acid, phosphoric acid, alkyl or aryl or succinimidyl carbonate, halide, or thioester functions (possibly substituted with appropriate protecting groups that can be removed before further chemical reaction). In some cases, additional reagents are added during the coupling reactions to begin or enhance the covalent attachments. The reagents and synthetic techniques needed for the coupling of D to L are well known to one of ordinary skill in the art. In cases when both functional groups X₅ and X₄ are present on L during a coupling reaction, X₄ is chemically protected to prevent reaction with D.

The preparation of the pharmaceutical agents and analogs is accomplished using techniques well know to those of ordinary skill in the art.

In a preferred embodiment, functional group, X₅ on L, is the p-nitrophenyl ester on dipeptide, H-Cbz-Gly-Phe-ONp and the reactive substituent on D is the amino group on the cancer drug analog H-Gly-Gly(\tilde{□}5-fluorouracil)-OEt. The covalently coupled amide product D-L is shown in Reaction Scheme 9 below.

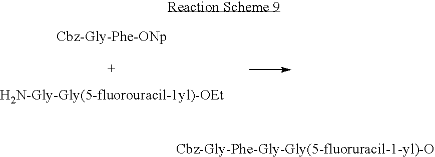

In another preferred embodiment, X₅ is the p-nitrophenyl ester of poly(PEG2K-Lysine-Gly-Phe-Leu-Gly-ONp) (Compound 5) and the reactive functionality on D is the amine group on doxorubicin. The covalently coupled amide product, regular repeating linear polymer poly[D-L-M-P], (Compound 6) is shown in Reaction Scheme 10 below.

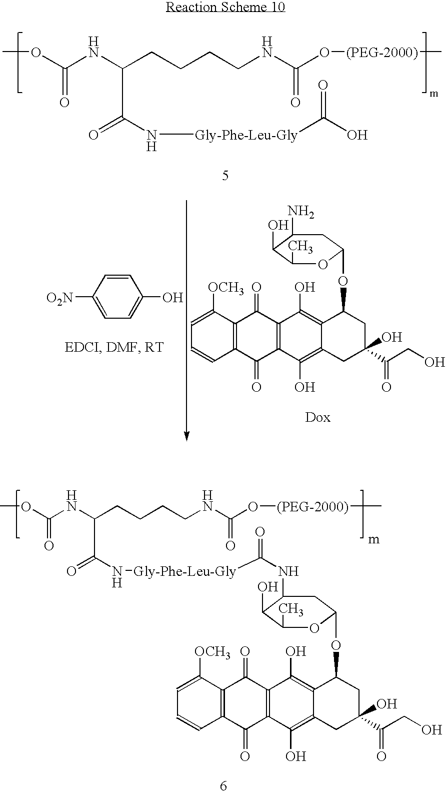

In another preferred embodiment, X₅ is the carboxylic acid function of polymeric peptide conjugate Compound 9 (poly[L-M-P]) and the reactive function on D is the unprotected amine of Compound 10 which will be later used to chelate a cancer treating platinum agent. The activating reagents 1-[3-(dimethylamino)propyl]-3-ethylcarbodiimide (EDCI) and 1-hydroxybenzotriazole (HOBt) are added to the reaction mixture enabling the coupling reaction to take place. The covalently coupled amide product (Compound 11) is shown in Reaction Scheme 11 below.

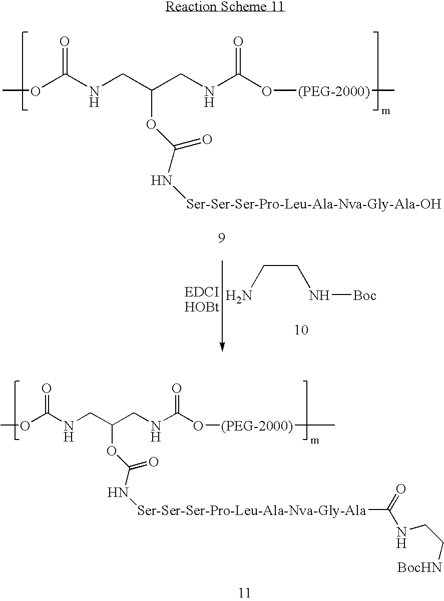

Attachment of Water-Soluble Polymer Segment, P to the Multifunctional Chemical Moiety, M

The water-soluble polymer segment, P, consists of a water soluble polymer or co-polymer system which contains at least two chemical functionalities for covalent attachment to the monomer, M. The reagents and synthetic techniques needed for the coupling of M to P are well known to those of ordinary skill in the art of organic, peptide, or oligonucleotide synthesis. Preferably, P is polyethylene glycol, poly(vinyl alcohol), poly(2-hydroxyethyl methacrylate), poly(acrylic acid), poly(methacrylic acid), poly(maleic acid) or analogs and combinations thereof. The preparation of the various polymers and analogs are accomplished using standard techniques well known to those of ordinary skill in the art.

In a preferred embodiment, groups X₁ and X₂ on M, are both amino functions (Compound 19) and the polymer, P, is an N-hydroxysuccinimidyl carbonate substituted analog of polyethylene glycol-2000 (Compound 8). The resulting biscarbamate conjugate with regular repeating linear co-polymer, P-M, (Compound 20) is shown in Reaction Scheme 12 below.

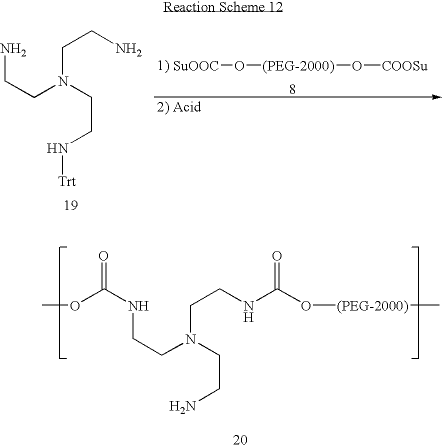

In another preferred embodiment, X₁ and X₂ are both amino groups on L-lysine and the reactive functionality on P are thiocarbonylimidazole groups (Compound 22). (See Reaction Scheme 13 below.) A base, sodium carbonate, is added to the reaction mixture to initiate the covalent coupling and produce the product, regular repeating linear co-polymer, poly[M-P] (Compound 23).

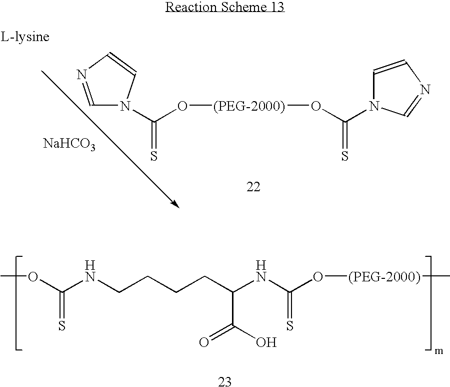

Attachment of Common Multifunctional Moiety, Q, to Polymer Segment, P

The attachment of common multifunctional moiety, Q, to polymer segment, P is accomplished using synthetic reactions similar to those for the conjugation of M to P.

Attachment of Linker, L to the Multifunctional Chemical Moiety, M

The coupling of the linker, L, to the multifunctional chemical moiety, M, is accomplished using the functions X₄ on L and X₃ on M. Substituents X₃ and X₄ are independently selected from or derived from hydroxyl, amino, thiol, alkyl or aryl disulfide, isothiocyanate, aldehyde, ketone, carboxylic acid, sulfonic acid, phosphoric acid, alkyl or aryl or succinimidyl carbonate, halide, or thioester functions (possibly substituted with appropriate protecting groups that can be removed before further chemical reaction). The reagents and synthetic techniques needed for the coupling of L to M are well known to those of ordinary skill in the art.

5.7 Mechanisms of Drug Release

The biologically active agent, D, is covalently coupled to the polymeric drug conjugates poly[D-L-M-P] and Q(-P-L-D)ₖ via an enzymatically cleavable bond present in the linker, L. The rate of release of D from either conjugate will depend on the mechanism of cleavage in vivo. D can be cleaved from the constructs by biological or physiological processes, or by chemical reactions. D will be released either directly from the polymeric drug conjugate or in the form of a complex D-L' (a compound containing D coupled to all or a part of L). The release of D may involve a combination of both enzymatic and non-enzymatic processes.

Cleavage (either with a single or multiple steps) resulting in the release of the active agent D may be brought about by non-enzymatic processes. For example, chemical hydrolysis (e.g., at an ester bond) may begin by simple hydration of the poly[D-L-M-P] conjugate upon delivery to the organism. Hydrolytic cleavage may result in the release of the complex L-D or the free active compound D. Cleavage can also be initiated by pH changes. For instance, the prodrug conjugate poly[D-L-M-P] may be dissolved in a minimally buffered acidic or basic pH solution before delivery. The bond between L and D or M and L of the prodrug conjugate poly[D-L-M-P] would then be characterized by a high degree of chemical lability at a physiological pH of 7.4, and would thus be cleaved when the conjugate is delivered to the tissue or circulatory system of the organism, releasing either the active agent D or the L-D complex. If necessary, a second reaction, either chemical or enzymatic, would result in the cleavage of D from the L-D complex. It is well known to those skilled in the art that N-Mannich base linkages exhibit this type of activity.

Cleavage can also occur due to an oxidative/reductive reaction. For example, a disulfide linkage can be created between L and D or M and L of the prodrug conjugate poly[D-L-M-P]. Such prodrug complexes would be stable at physiological pH. The bond between L and D or M and L of the prodrug conjugate poly[D-L-M-P] would be characterized by a high degree of chemical lability in reducing environments, such as in the presence of glutathione. If necessary, a second reaction, either chemical or enzymatic, would result in the cleavage of D from the L-D complex.

Proteolytic enzymes are produced in or near diseased tissues and organs as a result of biological signals from infectious agents, blood-borne cytokines, diseased tissue itself, or fluids near diseased tissue. According to the present invention, the linking group L is designed to be cleaved between L and D or M and L of the conjugate poly[D-L-M-P]. Such proteolytic enzymes can result from either the treated organism, or from microbial infection. Examples of such enzymes include, but are not limited to: metalloproteinases and other extracellular matrix component proteases (including collagenases, stromelysins, matrilysin, gelatinases and elastases), lysosomal enzymes (including cathepsin), serine proteases and other enzymes of the clotting cascade (such as thrombin), enzymes of the endoplasmic reticulum (such as cytochrome P450 enzymes, hydrolytic reaction enzymes and conjugation reaction enzymes), non-specific aminopeptidases and esterases, carboxypeptidases, phosphatases, glycolytic enzymes, and other enzymes that are present during certain disease conditions (such as angiotensin converting enzyme). Thrombin-like, alanine aminopeptidase, and elastase-like enzymatic activity are common in bacterial infections, and the amino acid cleavage sequences of such enzymes are well-documented.

There are many examples of possible amino acid sequences which can be used to cleave the linking group L at or near the site of diseased tissue in the constructs poly[D-L-M-P] and Q(-G-L-D)ₖ. For example, thrombin (a serine protease that is activated during the clotting cascade) cleaves the Arg-Gly bond in the following sequences:

-Gly-Arg-Gly-Asp-

-Gly-Gly-Arg-

-Gly-Arg-Gly-Asp-Asn-Pro

-Gly-Arg-Gly-Asp-Ser

-Gly-Arg-Gly-Asp-Ser-Pro-Lys

Matrix metalloproteinases (MMPs) and other extracellular matrix proteases are prevalent in healing and metabolism. However, this enzyme family is also implicated in various pathological processes, including chronic inflammation, arthritis, and cancer. In particular, MMPs are active during tumor growth and are necessary for metastasis. One major extracellular protein is collagen, which has a characteristic repeat amino acid sequence: -Gly-Pro-Y-Gly-Pro-Z (where Y and Z are any amino acids, except Pro or Hypro and X is any amino acid or organic compound). Matrix metalloproteinases and other extracellular matrix proteases cleave primarily at Leu-Gly or Ile-Gly bonds. Amino acid sequences which are cleaved by this family of enzymes include, but are not limited to:

-Gly-Pro-Asn-Gly-Ile-Ala-Gly-Asn-

-Gly-Pro-Gln-Gly-Ile-Ala-Gly-Asn-

-Gly-Pro-Asn-Gly-Ile-Phe-Gly-Asn-

-Gly-Pro-Leu-Gly-Val-Arg-Gly-

-Gly-Pro-Leu-Gly-Met-Phe-Ala-Thr-

-Pro-Leu-Gly-Leu-Trp-Ala-

-Pro-Leu-Ala-Nva-Gly-Ala-

-Pro-Leu-Gly-Leu-Gly-Ala-

-Gly-Pro-Tyr-Ala-Pro-Ala-Gly-His-

-Gly-Pro-Asn-Gly-Ile-Leu-Gly-Asn-

-Pro-Leu-Gly-Met-Leu-Ser-

-Leu-Ile-Pro-Val-Ser-Leu-Ile-Ser-

-Gly-Pro-Leu-Gly-Pro-Z

-Gly-Pro-Ile-Gly-Pro-Z

-Pro-Leu-Gly-Pro-D-Arg-Z

-Ala-Pro-Gly-Leu-Z

-Pro-Leu-Gly-(Sleu)-Leu-Gly-Z

-Pro-Gln-Gly-Ile-Ala-Gly-Trp-

-Pro-Leu-Gly-Cys(Me)-His-

-Pro-Leu-Gly-Leu-Trp-Ala-

Other sequences which are cleaved by this family of enzymes include, but are not limited to:

-Pro-Leu-Ala-Leu-Trp-Ala-Arg- (human Fibroblast Collagenase)

-Pro-Leu-Ala-Tyr-Trp-Ala-Arg- (human Neutrophil Collagenase)

-Pro-Tyr-Ala-Tyr-Trp-Met-Arg- (human Fibroblast Stromelysin)

-Pro-Leu-Gly-Met-Trp-Ser-Arg- (human Fibroblast or Neutrophil Gelatinases)

-Ala-Ala-Ala- (elastase)

-Ala-Ala-Pro-Ala- (elastase)

-Ala-Ala-Pro-Val- (elastase)

-Ala-Ala-Pro-Leu- (elastase)

-Ala-Ala-Pro-Phe- (elastase)

-Ala-Tyr-Leu-Val- (elastase)

Another example of an enzyme that is up-regulated due to disease, and therefore can be exploited according to the present invention, is the angiotensin converting enzyme. This enzyme cleaves at amino acid sequences which include, but are not limited to:

-Asp-Lys-Pro-

-Gly-Asp-Lys-Pro-

-Gly-Ser-Asp-Lys-Pro-

Another example of an enzyme that can be exploited according to the present invention is plasmin. This enzyme cleaves amino acid sequences which include, but are not limited to:

-Ala-Phe-Lys-

-Nle-HHT-Lys- (HHT=hexahydrotyrosine)

Another example of an enzyme that can be exploited according to the present invention is urokinase plasminogen activator. This enzyme cleaves amino acid sequences which include, but are not limited to:

-Glu-Gly-Arg-

-Pro-Gly-Arg-

Cys-Pro-Gly-Arg-

□-Ala-Gly-Arg-

Another example of an enzyme that can be exploited according to the present invention is furin. This enzyme cleaves amino acid sequences which include, but are not limited to:

-Arg-X-X-Arg-

Cells at the site of diseased tissue will produce numerous enzymes, growth factors, and cytokines that are present elsewhere in the organism at much lower concentrations. For example, cells that are involved with inflammation that produce secreted and cell-surface enzymes include: granulocytes (neutrophils, eosinophils, basophils), monocytes/macrophages, and lymphocytes. Activated macrophages are known to secrete elastase, collagenase and other MMPs, plasminogen activator, and other proteolytic enzymes. Activated peritoneal macrophages are known to produce hydrogen peroxide, which can be used to cleave D from the invented prodrug conjugates. Eosinophils, activated at the site of inflammation, produce lysosomal enzymes, peroxidase, histaminase, and other enzymes. As another example of disease-specific cleavage enzymes, various cancer cells (e.g., from prostate tumors) produce secreted or cell-surface enzymes that cleave specific amino acid sequences.

The linker may also comprise a peptide sequence which can be cleaved by aspartic proteinases such as pepsin, chymosin, lysosomal cathepsins D, processing enzymes such as renin, and certain fungal protease (penicillopepsin, rhizopuspepsin, endothiapepsin), and viral proteinases such as the protease from the AIDS virus (HIV).

The invention now being generally described, it will be more readily understood by reference to the following examples which are included merely for purposes of illustration of certain aspects and embodiments of the present invention, and are not intended to limit the present invention in any way.

### 6. EXAMPLES

6.1 Assembly of 5-Fluorouracil Linked Polymer Conjugate, 3

The present example (see Synthetic Pathway 1 below) describes the preparation of a regular repeating linear polymeric drug conjugate of the invention in which the cancer treatment agent, D, is 5-fluorouracil, the enzymatically cleaved region of the linker, (L₁-Lₙ), is an analog of the tetrapeptide, -Gly-Phe-Gly-Gly-, the multifunctional chemical moiety, M, is L-lysine, and the water soluble polymer segment, P, is poly(ethylene glycol) with an average MW of about 2000 (PEG-2000).

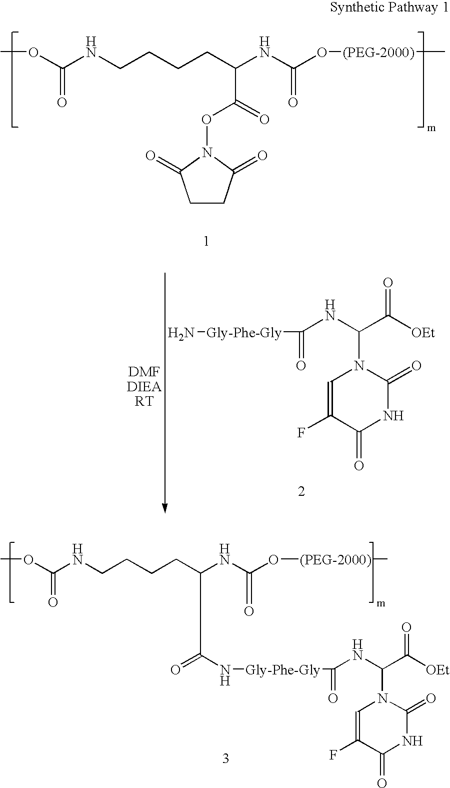

Preparation of Polymeric Prodrug Construct, 3

A clean dry 20 mL reaction vial equipped with a magnetic stir bar, was charged with 6 mL of dichloromethane, 3 mL of tetrahydrofuran, 309 mg (0.58 mmol) of glycyl-L-phenylalanylglycyl-2(R,S)-(5-fluorouracil-yl)glycine ethyl ester, 2, (prepared by the method of M. Nichifor and E. H. Schacht, Tetrahedron 50 (1994) 3747-3760), 1.168 g (0.508 mmol) of poly (PEG-Lys-NHS), 1, (NJ Center for Biomaterials, Piscataway, N.J.), and 0.204 mL (1.17 mmol) of N,N-diisopropylethyl amine(DIEA). The resultant mixture was magnetically stirred at room temperature for 3.5 h under argon and then the solvent was evaporated under reduced pressure. The distillation residue was dissolved in 30 mL of 0.01% acetic acid (pH 4) and dialyzed (Spectrum, Rancho Domingues, Calif., Spectra/Por 7 dialysis membrane, 3500 MWCO) against 4 L of 0.01% acetic acid (pH 4) for 52 h. The contents of the dialysis tubing was lyophilized to obtain 530 mg (71% yield) of polymeric prodrug construct, 3, as a white hygroscopic solid. UV analysis at 265 nm determined that the product contained 4.0% 5-fluorouracil. C-18 HPLC (YMC Pack DDS-AP colymn, AP-302, 150×4.6 mm I.D., S-5 □m, 30 nm, using a 50%-100% water:methanol gradient over 20 min) showed the product purity to be 99.7%

6.2 Assembly of Doxorubicin Linked Polymer Conjugate, 6

The present example describes the preparation (see Synthetic Pathway 2) of a regular repeating linear polymeric drug conjugate in which the chemical treatment agent, D, is doxorubicin, the enzymatically cleaved region of the linker, (L₁-Lₙ), is the tetrapeptide, Gly-Phe-Leu-Gly, the multifunctional chemical moiety, M, is L-lysine, the water soluble polymer segment, P, is poly(ethylene glycol) with an average MW of about 2000 (PEG-2000).

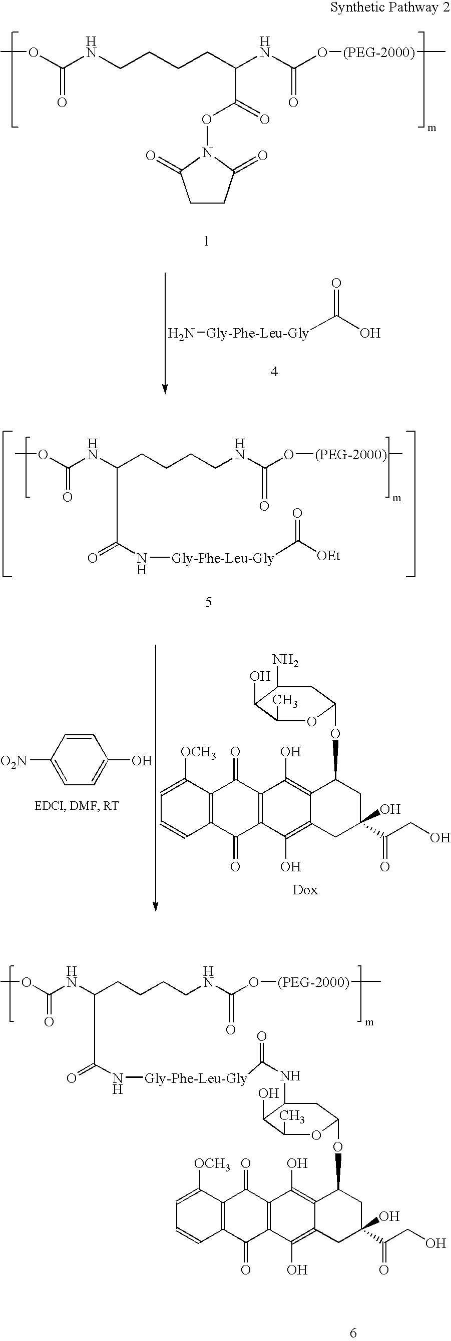

Preparation of Polymeric Prodrug Construct, 6

A 20 mL reaction vial equipped with a magnetic stir bar, was charged with 36.3 mg (0.0158 mmol) of poly (Peg-Lys-OSu), 1, (NJ Center for Biomaterials), 8.4 mg (0.0166 mmol) of H-Gly-Phe-Leu-Gly-OH.TFA (prepared using an Applied Biosystems Model 433A Peptide Synthesizer, HBTU/HOBt coupling & Fmoc AA protection), 0.2 mL of N,N-dimethylformamide (DMF), 0.2 mL of dichloromethane (DCM), and \tilde{□}□□L (0.0166 mmol) of □,N-diisopropylethylamine. There resultant reaction mixture was stirred at R.T. for 2 h and then the solvents were removed at reduced pressure. The distillation residue was dissolved in \tilde{□}□L of DMF and then 9.2 mg (0.0158 mmol) of doxorubicin hydrochloride and 3.0 mg (0.0158 mmol) of N,N-diisopropylethylamine (EDCI) was added. The reaction mixture was allowed to stir at RT for 18 h and the solvents were evaporated under reduced pressure. The distillation residue was dialyzed (Spectra/Por 7 dialysis membrane, 3500 MWCO) against 4 L of water for 72 h. The contents of the dialysis tubing were lyophilized to a give a red solid which was dissolved in 2 mL of dichloromethane and poured onto a pad of silica gel (Merck, 250-400 mesh, 6″ high×1″ diameter) that had been slurried in 9% MeOH in DCM. The silica gel pad was washed with 500 mL of 9% MeOH in DCM, 500 mL of 25% MeOH in DCM, and then 500 mL of 40% MeOH in DCM. The eluent that was collected from both the 25% and 40% MeOH in DCM washes were combined and the solvents were removed under reduced pressure to give prodrug construct, 6, as a dark red solid. UV analysis at 480 nm determined that the product contained 13.3% doxorubicin. C-18 HPLC (YMC Pack DDS-AP colymn, AP-302, 150×4.6 mm I.D., S-5 □m, 30 nm, using a 50%-100% water:methanol gradient over 20 min) showed the product purity to be 99.1%

6.3 Assembly of Drug Linked Polymer Conjugate, 13

The present example describes the preparation (see Synthetic Pathway 3) of a regular repeating linear polymeric drug conjugate of the invention in which the pharmaceutical agent, D, is a chelated ethylenediamine platinum dichloride complex, the enzymatically cleaved region of the linker, (L₁-Lₙ), is derived from the peptide, Ser-Ser-Ser-Pro-Leu-Ala-Nva-Gly-Ala, the multifunctional chemical moiety, M, is 1,3-diamino-2-propanol, and the water soluble polymer segment, P, is poly(ethylene glycol) (PEG-2000) with an average MW of about 2000.

Preparation of doubly tBoc-protected 1,3-diamino-2-propanol (DBDAP)

A 1 L three neck round bottom flask equipped with a magnetic stir bar and thermometer was charged with 10.453 g of 1,3-diamino-2-propanol, 238 mL of 1.0N aqueous potassium hydroxide solution, 250 mL of tetrahydrofuran and 50.624 g of di-tert-butyl dicarbonate. The reaction mixture was stirred at RT for 18 h, the volume was reduced by half using rotary evaporation and 500 mL of ethyl acetate was added. The organic layer was separated from the aqueous layer and the organic layer was washed with 3×100 mL of 0.5N hydrochloric acid and 1×200 mL of saturated aqueous sodium chloride solution. The ethyl acetate solution was dried over anhydrous magnesium sulfate, filtered, and the solvent was removed by rotary evaporation to give 73.9 g (100% yield) of colorless syrup that solidified upon drying under vacuum 0.2 mm Hg) for 48 h. TLC on silica gel plates (5×10 cm, Mecrk, Darmstadt, Germany) using 7% methanol in chloroform as eluent showed one spot at R_{f}=0.42.

Preparation of NPC-DBDAP

A 1 L three neck round bottom flask equipped with a magnetic stir bar and thermometer was charged with 17.70 g (60.96 mmol) of DBDAP, 350 mL of dichloromethane, 4.93 mL (60.96 mmol) of pyridine and 12.29 g (60.96 mmol) of p-nitrophenylchlorocarbonate. The reaction mixture was stirred at RT under an argon atmosphere for 24 h, and then 200 mL of dichloromethane was added. The resultant solution was washed with 2×250 mL of 200 mM hydrochloric acid and 1×250 mL of saturated sodium chloride solution. The organic layer was dried over anhydrous sodium sulfate, and the solvents were removed by rotary evaporation to give 27.63 g (99% yield) of NPC-DBDAP.as a yellow waxy solid. The product was determined to be approximately 85% pure by TLC and was used without further purification. TLC on silica gel plates (5×10 cm, Mecrk, Darmstadt, Germany) using 7% methanol in chloroform as eluent.)

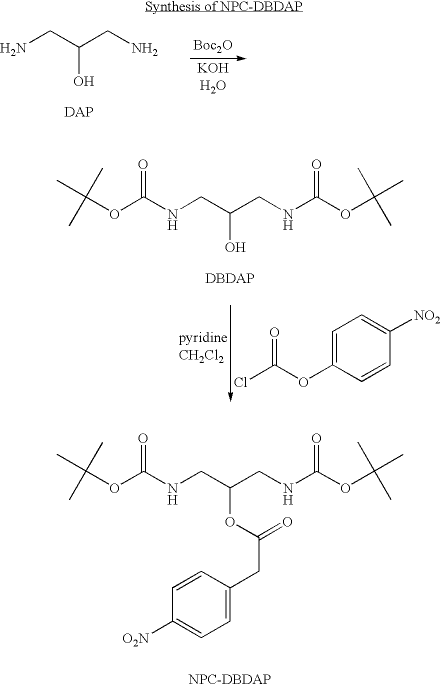

Synthesis of Peptide Analog, 7

Using Fmoc AA protection chemistry, H-Ser-Ser-Ser-Pro-Leu-Ala-Nva-Gly-Ala- was prepared on Wang solid phase synthesis resin using an Applied Biosystems Model 433A.

The peptide conjugate on resin was removed from the synthesizer and added to a three neck 250 mL round bottom flask which was equipped with a magnetic stir bar, thermometer, and argon gas inlet-outlet. Then, 75 mL of 1-methyl-2-pyrrolidinone (NMP), 1.82 g of NPC-DBDAP and 697 mL of N,N-diisopropylethylamine (DIEA) was added, the reaction slurry was allowed to stir at RT for 16 h and an additional 1.82 g of DBDAP was then added. The solids were filtered, washed with 3×150 mL of NMP and 3×50 mL of dichloromethane. The filter cake was dried at RT/0.2 mm Hg for 16 h and the peptide analog was cleaved from the Wang resin using a mixture of trigluoroacetic acid, triisopropyl silane and water as described in the peptide ABI synthesizer protocols. The peptidic product, 7, was isolated by preparative HPLC using an acetonitrile:water: 0.1% TFA elution gradient on a 20 cm×2 cm RP-18 Waters HPLC column.

Preparation of polymer-peptide conjugate, 9

A clean dry 50 mL three neck round bottom flask equipped with a magnetic stir bar, thermometer and argon inlet-outlet was charged with 14.5 mL of N,N-dimethylformamide (DMF), 1.075 g of peptide analog, 7, 1.806 g of PEG-2000 bis OSu carbonate, 8, and 0.794 mL of triethylamine. The reaction mixture was stirred for 18 h at RT and it was then dropped slowly onto 500 mL of vigorously stirred diethyl ether. The resultant precipitates were filtered, washed with 100 mL of diethyl ether, and the filter cake was dissolved in 40 mL of 0.5N hydrochloric acid. The hydrochloric acid solution was then dialyzed (Spectrapor 7 dialysis tubing with MWCO 3500) against 4×4 L of deionized water for 18 h to give 2.335 g (97% yield) of polymer-peptide conjugate, 9, as a white hydroscopic powder.

Preparation of Ethylene Diamine Substituted Polymer-Peptide Conjugate, 12

A clean dry 50 mL three neck round bottom flask equipped with a magnetic stir bar, thermometer and argon inlet-outlet was charged with 2.242 g (0.758 mmol) of 9, 0.265 g (0.834 mmol) of mon-N-(9-fluorenylmethoxycarbonyl)-ethylenediamine, 10, 0.123 g (0.910 mmol) of O-Benzotriazol-1-yl-N,N,N′,N′-tetramethyluronium hexafluorophosphate (HBTU), 25 mL of DMF, 0.160 g (0.834 mmol) of EDCI, and 0.291 mL (1.668 mmol) of DIEA. The reaction mixture was stirred for 30 h at RT and it was then dropped slowly onto 600 mL of vigorously stirred diethyl ether. The resultant precipitates were filtered, washed with 100 mL of diethyl ether, and the filter cake was dissolved in 40 mL of DMF and 6 mL of piperidine. The reaction mixture was stirred at RT for 1.5 h and it was then dropped slowly onto 1 L of vigorously stirred diethyl ether. The resultant precipitates were filtered, washed with 100 mL of diethyl ether, dried under vacuum (0.2 mm Hg) for 48 h. The solids were then dissolved in 25 mL of DMF, the resultant solution was dropped slowly into 700 mL of vigorously stirred diethyl ether, the precipitates were filtered, washed with 100 mL of diethyl ether and dried under vacuum (0.2 mm Hg) for 48 h to give 2.3 g (100% yield) of ethylenediamine substituted polymer-peptide conjugate, 12. Analytical HPLC indicated the material to be of >99% purity.

Preparation of Platinum Chelated Polymer Construct, 13

A clean 50 mL three neck round bottom flask equipped with a magnetic stir bar and thermometer was charged with 0.313 g (0.754 mmol) of platinum tetrachloroplatinate (II), 17 mL of water, and 2.273 g (0,754 mmol) of polymer-peptide conjugate, 12. The reaction mixture was stirred at RT for 21 h and then 0.300 mL of saturated aqueous sodium bicarbonate solution was added. The reaction mixture was allowed to stir for an additional 72 h and the pH was then adjusted to between 6.5 using saturated aqueous sodium bicarbonate solution. The reaction mixture was then filtered through Centriplus YM-10 filtration tubes (MWCO=10,000) to obtain a colorless aqueous solution. The solution was then frozen and lyophilized to give 1.97 g (80% yield) of platinum chelated polymeric construct, 13, as a light brown solid.

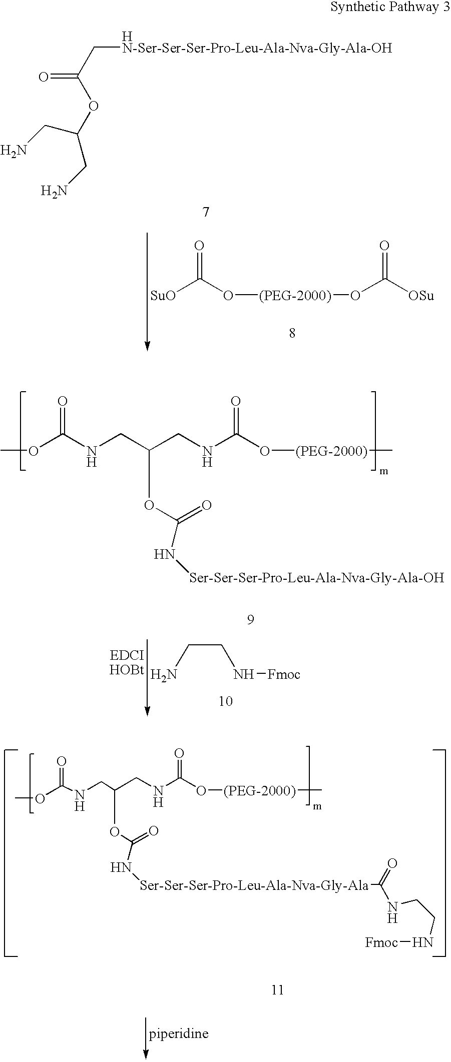

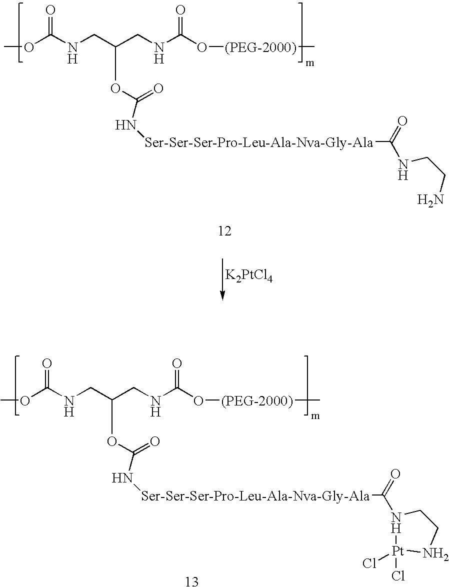

6.4 Assembly of Drug Linked Polymer Conjugate, 17

The present example describes the preparation (see Synthetic Pathway 4) of a regular repeating linear polymeric drug conjugate of the invention in which the pharmaceutical agent, D, is dichloro(1,2-diaminocyclohexane)platinum (II); the enzymatically cleavable region of the linker, (L₁-Lₙ), is peptide, Ser-Ser-Ser-Gly-Pro-Asn-Gly-Ile-Ala-Gly-Asn-Asp, the multifunctional chemical moiety, M, is 1,2-diamino-2-propanol, and the water soluble polymer segment, P, is poly(ethylene glycol) with an average MW of about 2000.

Synthesis of Peptide Analog, 14

Using Fmoc AA protection chemistry, H-Ser-Ser-Ser-Gly-Pro-Asn-Gly-Ile-Ala-Gly-Asn-Asp-SPSR was prepared on Wang solid phase synthesis resin using an Applied Biosystems Model 433A. The peptide conjugate on resin was removed from the synthesizer and added to a three neck 250 m/L round bottom flask which was equipped with a magnetic stir bar, thermometer, and argon gas inlet-outlet. Then, 75 mL of 1-methyl-2-pyrrolidinone (NMP), 1.82 g of DBDAP and 697 mL of N,N-diisopropylethylamine (DIEA) was added, the reaction slurry was allowed to stir at RT for 18 h and an additional 1.82 g of DBDAP was then added. The solids were filtered, washed with 3×150 mL of NMP and 3×50 mL of dichloromethane. The filter cake was dried at RT/0.2 mm Hg for 16 h and the peptide analog was cleaved from the Wang resin using a mixture of trifluoroacetic acid, triisopropylsilane, and water as described in the peptide ABI synthesizer protocols. The peptidic product, 14, was isolated by preparative HPLC using an acetonitrile:water: 0.1% TFA elution gradient on a 20 cm×2 cm RP-18 Waters HPLC column.

Preparation of Polymer-Peptide Conjugate, 15

A clean dry 50 mL three neck round bottom flask equipped with a magnetic stir bar, thermometer and argon inlet-outlet was charged with 8.5 mL of N,N-dimethylformamide (DMF), 0.783 g (0.552 mmol) of peptide analog, 14, 1.124 g (0.492 mmol) of PEG-2000 bis OSu carbonate, 8, and 0.576 mL (4.138 mmol) of triethylamine. The reaction mixture was stirred for 18 h at RT and it was then dropped slowly onto 500 mL of vigorously stirred diethyl ether. The resultant precipitates were filtered, washed with 100 mL of diethyl ether, and the filter cake was dissolved in 15 mL of saturated aqueous sodium bicarbonate solution and 20 mL of water. The bicarbonate solution was then dialyzed (Spectrapor 7 dialysis tubing with MWCO 3500) against 4×4 L of deionized water for 23 h to give 1.10 g (68% yield) of polymer-peptide conjugate, 15, as a white hydroscopic powder.

Preparation of Diiodo (1,2-diaminocyclohexane) platinum (II)

A clean 50 mL three neck round bottom flask equipped with a magnetic stir bar, heating mantle, and thermometer was charged with 3.524 g (4.23 mmol) of potassium iodide, 26 mL of water, and 2.203 g (5.31 mmol) of potassium tetrachloroplatinate (II). The reaction mixture was gently warmed to 33° C. and after the temperature cooled back to RT, 0.581 g (5.09 mmol) of 1,2-diaminocyclohexane and 10 mL of water was added. A yellow precipitate formed and the reaction mixture was stirred for 3 h at RT and then at 4° C. for 16 h. The precipitates were filtered, washed with 3×25 mL of water, 2×25 mL of ethanol, 2×25 mL of diethyl ether, and the filter cake was dried at RT/02.mm of Hg for 48 h to give 2.743 g (92% yield) of diiodo(1,2-diaminocyclohexane)platinum (II).

Preparation of 1,2-diaminocyclohexane Platinum Reagent, 16

A clean 20 mL reaction vial equipped with a magnetic stir bar was charged under an argon blanket with 0.456 g (0.810 mmol) of diiodo(1,2-diaminocyclohexane)platinum (II), 7.3 mL of water, and 0.269 g (1.58 mmol) of silver nitrate. The reaction mixture was stirred in the dark at RT for 18 h and then at 65° C. for 3.5 h. The reaction mixture was then cooled to RT, filtered under an argon blanket, and the filter cake was dissolved in 8 mL of water. The solution was determined to be 107 mM in Pt. This solution of compound 16 was used without further purification.

Preparation of Platinum Chelated Polymeric Construct, 17

A clean 20 mL reaction vial equipped with a magnetic stir bar was charged under an argon blanket with 1.08 g (0.329 mmol) of 15 and 3.683 mL of 107 mM aqueous solution of 16. The reaction mixture was stirred in the dark at RT for 14 h, an additional 2 mL of water was added, the reaction temperature was increased to 38° C. for 1.5 h, and then 11 mL of buffer (220 mM NaCl, 180 mM H₂PO₄, pH 7.4) was added. After stirring at 38° C. for 72 h, the reaction mixture was cooled to RT and filtered through Millipore YM-10 Centriplus filters. The filtrate was frozen and lyophilized to give 1.126 g (96.1% yield) of platinum chelated polymeric construct, 17.

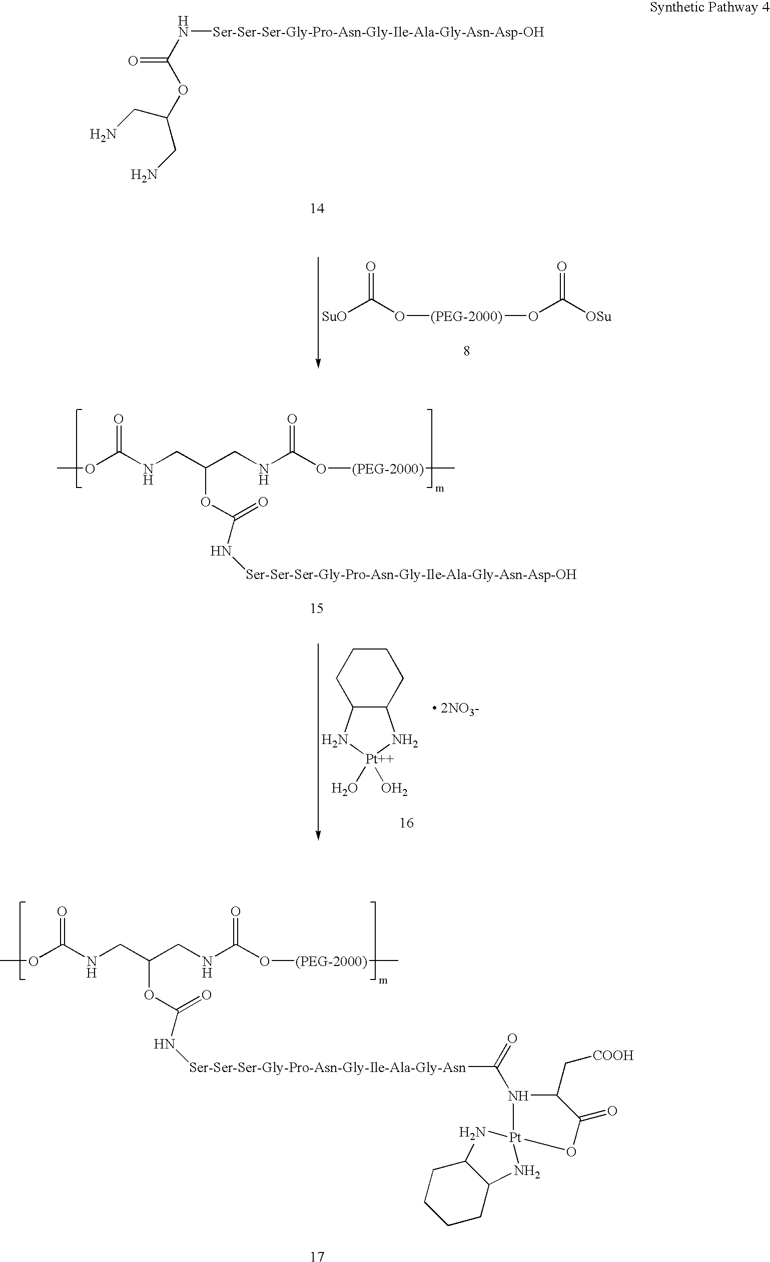

6.5 Assembly of Linear Block Co-Polymer, 20

The present example describes the preparation (see Synthetic Pathway 5) of a regular repeating linear polymeric backbone of the invention in which the multifunctional chemical moiety, M, is tris(2-aminoethyl)amine, and the water soluble polymer segment, P, is poly(ethylene glycol) (PEG-2000) with an average MW of about 2000.

Synthesis of Mono-Trityl Protected tris(2-aminoethyl)amine, 19

A clean dry 500 mL three neck round bottom equipped with a 250 mL addition funnel, thermometer, Drierite filled drying tube and magnetic stir bar was charged with 150 mL of dichloromethane, 13.01 g (88.96 mmol) of tris(2-aminoethyl)amine, 18, and 1.72 mL (9.88 mmol) of N,N-diisopropylethylamine. Then, 2.76 g (9.88 mmol) of triphenylmethyl chloride dissolved in 150 mL was added dropwise to the reaction mixture at room temperature. After 4 h, the reaction mixture was poured into a IL separatory funnel and washed with 2×150 mL of water. The organic layer was dried over anhydrous magnesium sulfate, filtered, the solvent was removed by rotary evaporation, and 100 mL of tert-butyl methyl ether was added to the distillation residue. Gaseous carbon dioxide was bubbled through the resultant solution for 1 h and the resultant precipitates were filtered, washed with 50 m/L of tert-butyl methyl ether, 50 mL of diethyl ether, and dried at 20° C./0.2 mm Hg for 24 h to yield 3.54 g (8.22 mmol, 83% yield) of mono-trityl protected tris(2-aminoethyl)amine, 19, as a white solid.

Synthesis of Co-Polymeric Backbone, 20

A clean dry 50 mL three neck round bottom equipped with a thermometer, Drierite filled drying tube and magnetic stir bar was charged with 15 mL of dichloromethane, 2.31 g (1 mmol) of PEG-2000 bis OSu carbonate, 8, and 527 mL (3 mmol) of N,N-diisopropylethylamine and 438 mg (1 mmol) of mono-trityl protected tris(2-aminoethyl)amine, 19. The reaction mixture was stirred at room temperature for 48 h, poured onto 100 mL of dichloromethane in a 250 mL separatory funnel, and washed with 50 mL of water. The organic layer was separated from the aqueous layer and the aqueous layer was extracted with 50 mL of dichloromethane. The organic layers were combined, dried over anhydrous magnesium sulfate and filtered. 7.5 mL of triisopropylsilane was added to the dichloromethane solution, and after 1 minute, 7.5 mL of trifluoroacetic acid was added. The reaction mixture was stirred at room temperature for 18 h during which time the yellow solution became colorless. The solvent was removed by rotary evaporation and 50 mL of ethyl acetate and 50 mL of water was added to the distillation residue. The organic layer was separated from the aqueous layer and the aqueous layer was washed with 2×50 mL of ethyl acetate. The aqueous solution was dialyzed against 4×4 L of distilled water over 2 days using Spectra/Por 7 dialysis tubing with a MWCO of 3500 (Spectrum, Rancho Dominguez, Calif.) and then lyophilized to give 750 mg of co-polymeric backbone, 20, as a white powder. GPC using PEG standards showed the material to have a M_{w} of 41,221 with a polydispersity of 1.87.

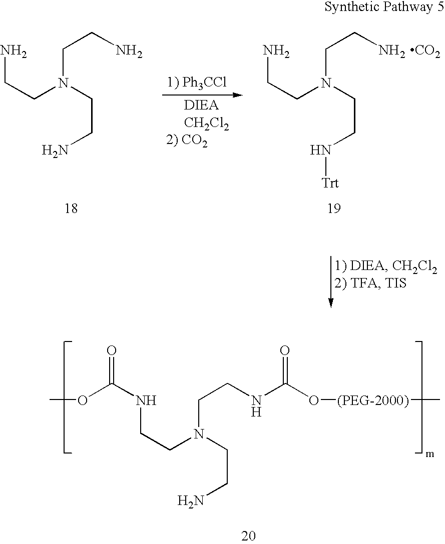

6.6 Assembly of Linear Block Co-Polymer, 23

The present example describes the preparation (see Synthetic Pathway 6) of a regular repeating linear polymeric backbone of the invention in which the multifunctional chemical moiety, M, is L-lysine, and the water soluble polymer segment, P, is poly(ethylene glycol) (PEG-2000) with an average MW of about 2000.

Synthesis of PEG-2000 bis-thioimidazole, 22

A clean dry 250 mL three neck round bottom equipped with a thermometer, Drierite filled drying tube and magnetic stir bar was charged with 100 mL of tetrahydrofuran, 33.71 g (16.86 mmol) of poly(ethylene glycol) with an average molecular weight of 2000 Da (Sigma-Aldrich, Milwaukee, Wis.) and 10.0 g of 1,1′-thiocarbonyldiimidazole (Sigma-Aldrich). diisopropylethylamine. The reaction mixture was stirred at room temperature for 16 h, the solvent was removed by rotary evaporation, and the distillation residue was dissolved in 90 mL of ethyl acetate. The ethyl acetate solution was slowly added by means of an addition funnel to 900 mL of vigorously stirred diethyl ether. The resultant mixture was allowed to stand for 16 h at 4° C., the precipitates were filtered, washed with 100 mL of diethyl ether, and the filter cake was dissolved in 150 mL of dichloromethane. The dichloromethane solution was added dropwise to 900 mL of vigorously stirred diethyl ether, and the resultant mixture was allowed to stand at 4° C. for 16 h. The precipitates were filtered, washed with 100 mL of diethyl ether, 100 mL of a 1:1 mixture of diethyl ether: hexane, and dried at 30° C. under vacuum to yield 32.8 g (88% yield) of PEG-2000 bis-thiocarbonylimidazole, 22, as a tan colored powder.

Synthesis of Linear Block Co-Polymeric Backbone, 23

A clean 500 mL three neck round bottom equipped with a thermometer and mechanical stirrer was charged with 384 mL of water, 0.795 g (5.4 mmol) of L-lysine, 1.416 g (16.86 mmol) of sodium bicarbonate and 12.08 g (5.4 mmol) of PEG-2000 bis-thiocarbonylimidazole, 22. The reaction mixture was stirred for 6 days at room temperature and then poured onto a mixture of 500 mL of 0.5N hydrochloric acid in saturated aqueous sodium chloride solution and 1.2 L of dichloromethane. The aqueous layer was separated from the organic layer, the aqueous layer was extracted with 1×200 mL of dichloromethane, the organic layers were combined, and washed with 1×200 mL of 0.5N hydrochloric acid in saturated sodium chloride solution. The dichloromethane solution was dried over anhydrous magnesium sulfate, filtered and the solvents were removed by rotary evaporation. The distillation residue was dissolved in 40 mL of dichloromethane and added dropwise to a vigorously stirred solution of 110 mL of hexanes in 640 mL of diethyl ether. The resultant mixture was allowed to stand at −20° C. for 16 h, the precipitates were filtered, washed with 2×40 mL of diethyl ether and dried at room temperature under vacuum for 72 h to yield 10.46 g (86.2% yield) of linear block co-polymer, 23, as a light tan solid, GPC using PEG standards showed the material to have a Mw of 25,780 with a polydispersity of 1.55.

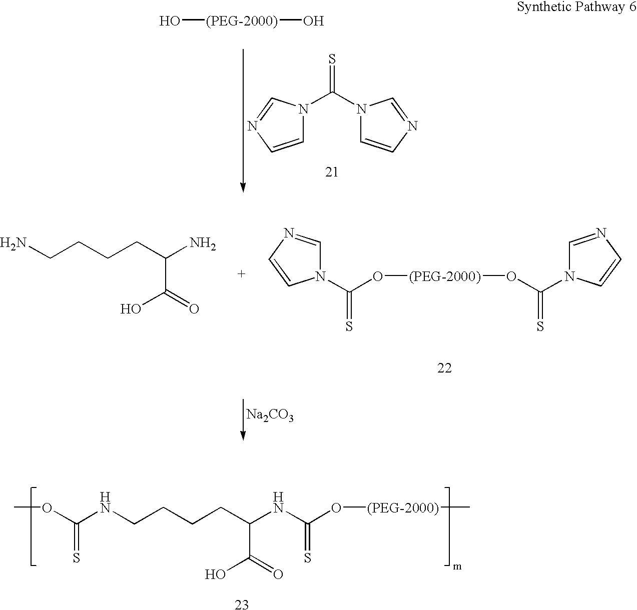

6.7 Assembly of Drug Linked Polymer Conjugate, 26

The present example (see Synthetic Pathway 7 below) describes the preparation of a regular repeating linear polymeric drug conjugate of the invention in which the pharmaceutical agent, D, is dichloro(1,2-diaminocyclohexane)platinum (II), the enzymatically cleaved region of the linker, (L₁-Lₙ), is peptide, -Ser-Ser-Ser-Ala-Phe-Lys-Asp-, the multifunctional chemical moiety, M, is L-lysine, and the water soluble polymer segment, P, is poly(ethylene glycol) with an average MW of about 2000 (PEG-2000).

Synthesis of Peptide, 24

Using Fmoc AA protection chemistry, H-Ser-Ser-Ser-Ala-Phe-Lys(iVDde)-Asp-OH, 14, was prepared on Wang solid phase synthesis resin using an Applied Biosystems Model 433A. The peptidic product was isolated by preparative HPLC using an acetonitrile: water: 0.1% TFA elution gradient on a 20 cm×2 cm RP-18 Waters HPLC column.

Preparation of Polymer-Peptide Conjugate, 25

A 100 mL reaction flask equipped with a magnetic stir bar, was charged with 3.908 g (1.70 mmol) of poly (Peg-Lys-OSu), 1, (NJ Center for Biomaterials), 2.0 g (1.70 mmol) of 24, 65 mL of N,N-dimethylformamide (DMF), 7 mL of water, and 1.482 mL (8.51 mmol) of N,N-diisopropylethylamine. The reaction mixture was stirred at R.T. for 48 h and then 2.0 mL of hydrazine was added. The resultant solution was stirred at RT for 1 h and it was then added dropwise to 1.0 L of anhydrous diethyl ether. The resultant precipitates were filtered, washed with 2×100 mL of a 1:1 mixture of diethyl ether:ethyl acetate and the filter cake was dried at RT under reduced pressure for 20 h. The dried solids were dissolved in 100 mL of 200 mM hydrochloric acid and dialyzed (Spectra/Por 7 dialysis tubing, 3500 MWCO) against 7×4 L portions of deionized water. The contents of the dialysis tubing were lyophilized to give 4.19 g (1.41 mmol, 83% yield) of polymer-peptide conjugate, 25 as a white solid.

Preparation of Platinum Chelated Polymeric Construct, 26

A clean 250 mL reaction flask equipped with a magnetic stir bar was charged with 4.074 g (1.377 mmol) of 25, 100 mL of water 2.3 mL of 500 nM sodium bicarbonate solution (to give a reaction mixture pH of 4.0-4.4), and 12.64 mL of a 109 mM aqueous solution of 16. The reaction mixture was stirred in the dark at RT for 20 h, and then 15 mL of a pH 3.93 phosphate buffered saline solution and 2.4 mL of 500 mM sodium hydrogen carbonate solution was added to bring the pH of the reaction mixture to approximately 5.0. The reaction mixture was stirred at 37° C. for an additional 44 h and centrifugally filtered through a 0.45 micron nylon membrane with a 10,000 Dalton MWCO. The filtrate was lyophilized to give 4.24 g (1.29 mmol, 94% yield) of construct, 26, as a yellow solid. Atomic absorption analysis showed this material to be 3.1% platinum by weight.

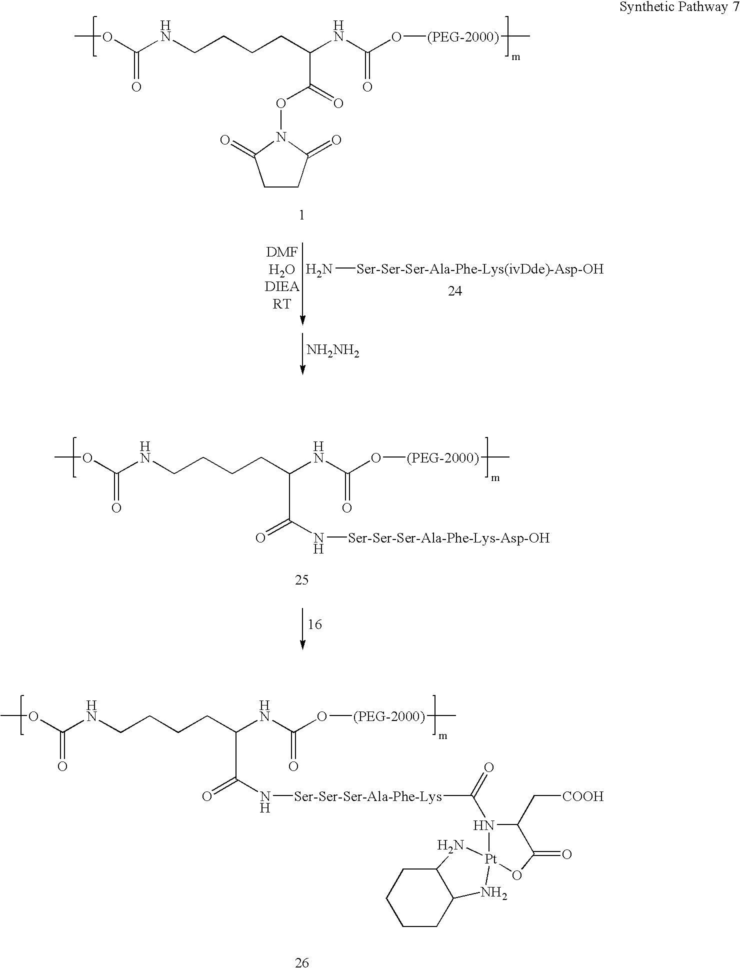

6.8 Assembly of Drug Linked Polymer Conjugate, 31

The present example (see Synthetic Pathway 8 below) describes the preparation of a regular repeating linear polymeric drug conjugate of the invention in which the pharmaceutical agent, D, is dichloro(1,2-diaminocyclohexane)platinum (II), the enzymatically cleaved region of the linker, (L₁-Lₙ), is the peptide, -Ser-Ser-Ser-Pro-Gly-Arg-Asp-, the multifunctional chemical moiety, M, is L-lysine, and the water soluble polymer segment, P, is poly(ethylene glycol) with an average MW of about 2000 (PEG-2000).

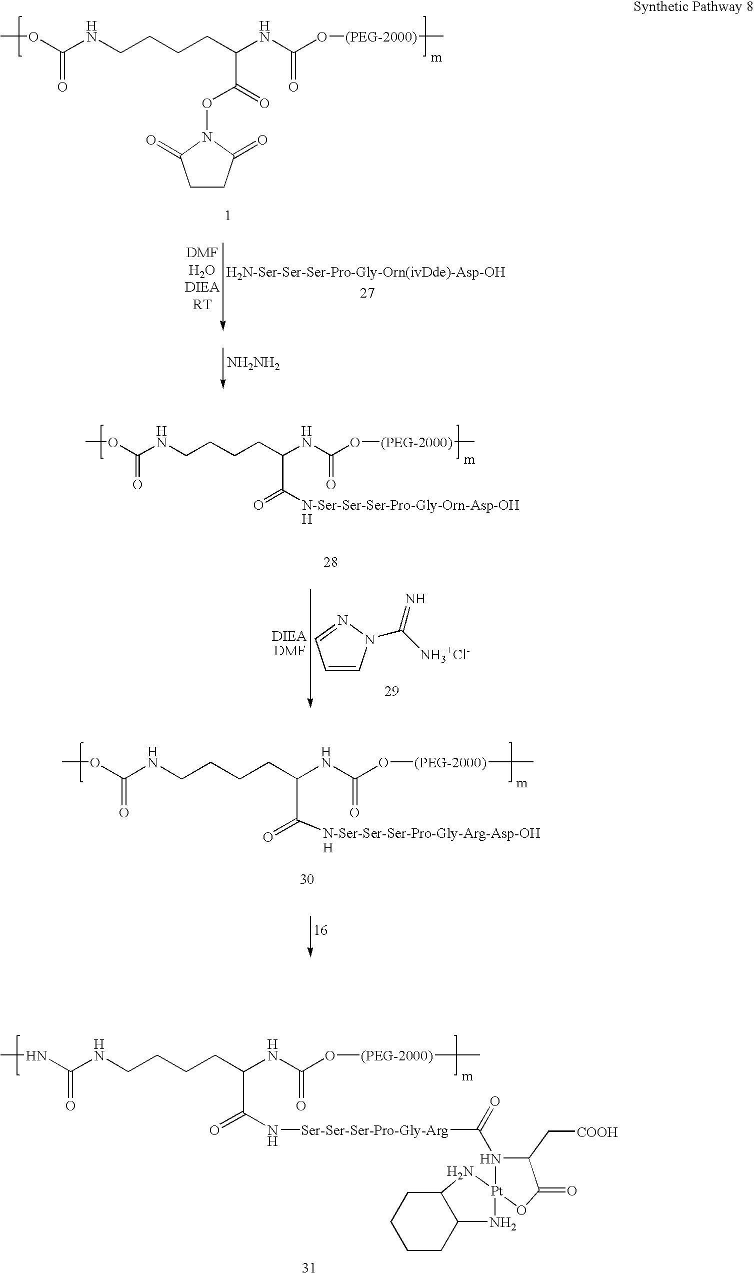

Synthesis of Peptide, 27

Using Fmoc AA protection chemistry, and HBTU/HOBt coupling reagents, H-Ser-Ser-Ser-Pro-Gly-Orn(ivDde)-Asp-OH, 27, was prepared on Wang solid phase synthesis resin using an Applied Biosystems Model 433A. The peptidic product was isolated by preparative HPLC using an acetonitrile: water: 0.1% TFA elution gradient on a 20 cm×2 cm RP-18 Waters HPLC column.

Preparation of Polymer-Peptide Conjugate, 28

A 20 mL reaction vial equipped with a magnetic stir bar, was charged with 1.677 g (0.731 mmol) of poly (Peg-Lys-OSu), 1, (NJ Center for Biomaterials), 0.609 g (0.657 mmol) of 27, 15 mL of N,N-dimethylformamide (DMF), and 0.891 mL (5.115 mmol) of N,N-diisopropylethylamine. The reaction mixture was stirred under argon at RT. for 72 h and it was then added dropwise to 650 mL of anhydrous diethyl ether. The resultant precipitates were filtered, washed with 100 mL of diethyl ether and the filter cake was dried at RT under reduced pressure for 20 h. The dried solids were dissolved in a solution of 0.70 mL of hydrazine in 25 mL of N,N-dimethylformamide and the resultant solution was stirred for 1.5 h. at RT. The reaction mixture was added dropwise to 1.0 L of diethyl ether, the precipitates were filtered, dried at RT under reduced pressure and dissolved in 30 mL of 250 mM hydrochloric acid and dialyzed (Spectra/Por 7 dialysis tubing, 3500 MWCO) against 5×4 L portions of deionized water over 27 h. The contents of the dialysis tubing were lyophilized to give 1.88 g (0.66 mmol, 90% yield) of polymer-peptide conjugate, 28 as a white solid.

Conversion of Polymer-Peptide Conjugate, 28 to Polymer-Peptide Conjugate, 30

A clean dry 20 mL reaction vial equipped with a magnetic stir bar was charged with 1.846 g (0.641 mmol) of 28, 13 mL of N,N-dimethylformamide, 0.893 mL (5.128 mmol) of DIEA, and 0.470 g of 29. The reaction mixture was stirred under argon at RT for 2 h and it was then added dropwise to 800 mL of diethyl ether. The resultant precipitates were filtered, washed with 100 mL of diethyl ether, dried at RT under reduced pressure, and dissolved in 40 mL of a 1:1 mixture of saturated sodium chloride solution in water. The solution was dialyzed (Spectra/Por 7 dialysis tubing, 3500 MWCO) against 5×4 L of deionized water over 27 h and the contents of the dialysis tubing was lyophilized to give 1.805 g (95% yield) of polymer-peptide conjugate, 30, as a white solid.

Preparation of Platinum Chelated Polymeric Construct, 31

A clean 100 mL reaction flask equipped with a magnetic stir bar was charged with 1.708 g (0.576 mmol) of 30, 37 mL of water, and 5.4 mL of a 128 mM aqueous solution of 16. The reaction mixture was stirred in the dark at RT for 15 h, and then 5.2 mL of a pH 4 phosphate buffered saline solution and 0.40 mL of a 1M sodium bicarbonate was added to bring the pH of the reaction mixture to approximately 5.0. The reaction mixture was stirred at 37° C. for an additional 20 h and it was centrifugally filtered through a 0.45 micron nylon membrane with a 10,000 Dalton MWCO. The filtrate was lyophilized to give 1.78 g (95% yield) of construct, 31, as a white solid. Atomic absorption analysis showed this material to be 2.86% platinum by weight.

6.9 In Vivo Studies

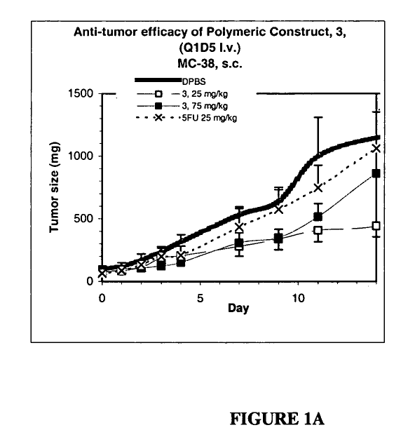

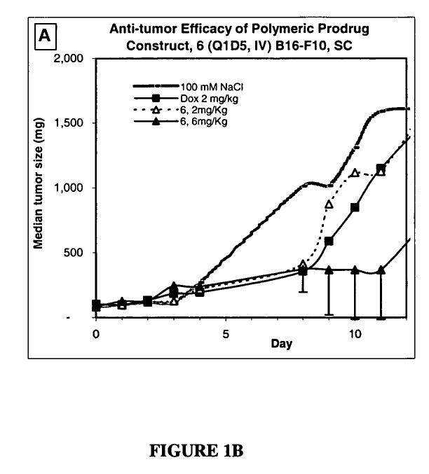

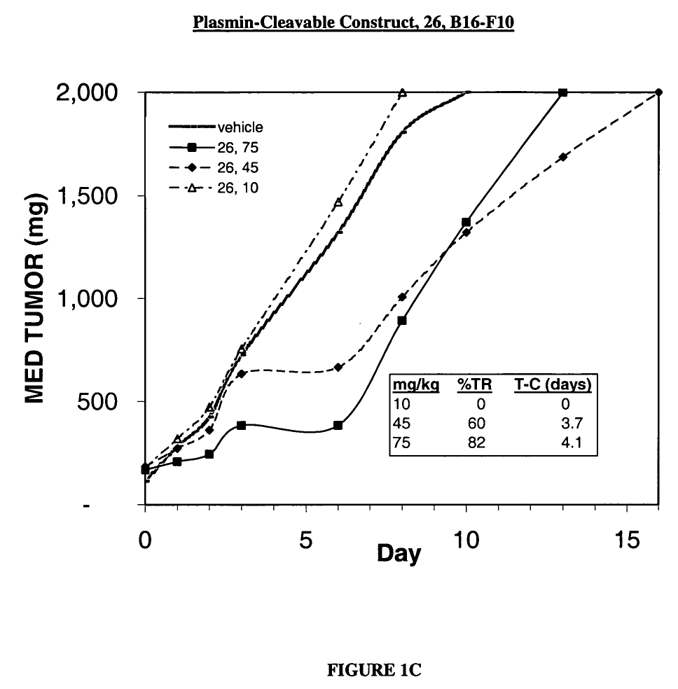

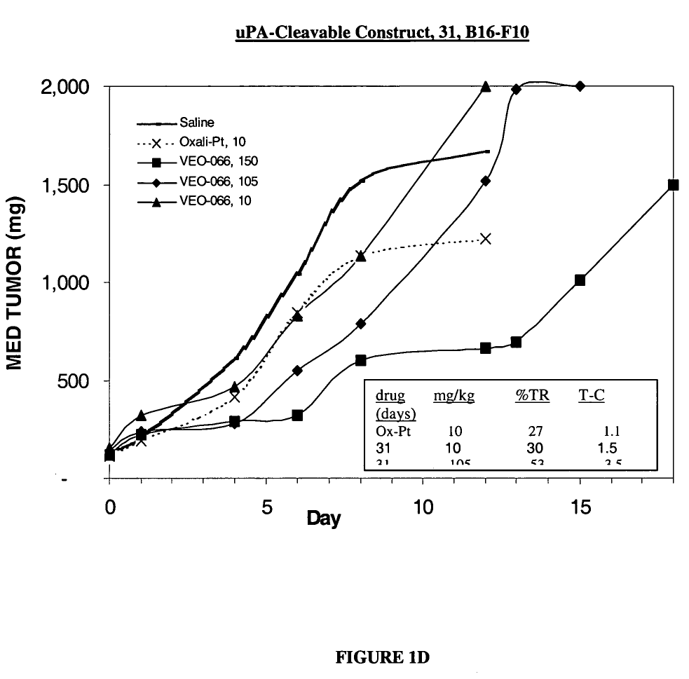

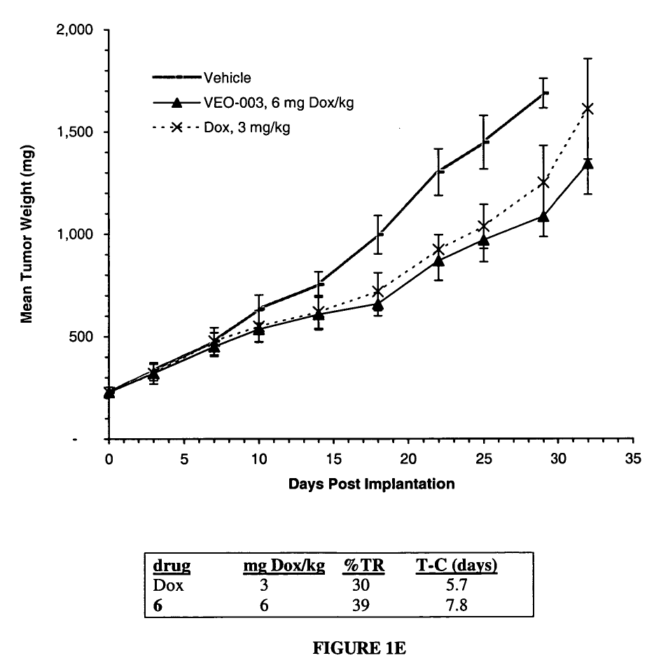

Polymeric prodrug conjugate, conjugate, 6, a doxorubicin (Dox)-containing construct in which the drug moiety is attached to the PEG backbone via a cathepsin B-cleavable linker peptide was tested in mice bearing murine melanoma B16-F10 implanted s.c. Mice were treated by daily i.v. (tail vein) injection for 5 consecutive days, beginning on the day when tumors became palpable. The conjugate was studied at the maximum tolerated dose (MTD) of the free drug, 2 mg Dox/kg (equimolar comparison), and at the conjugate's MTD, 6 mg Dox/kg (equitoxic comparison). The conjugate, 6, or Dox administered at the free drug's MTD (2 mg/kg) had minimal anti-tumor effect (30% tumor reduction, 3.4 days delay in reaching 500 mg). But at the conjugate's MTD of 6 mg Dox/kg there was substantial activity (FIG. 1A). The conjugate reduced tumor by 81% and extended the time to reach 500 mg to 6.5 days. Therefore as a polymeric prodrug conjugate it was possible to increase the tolerated drug dose by a factor of 3, and this increase permitted the achievement of substantially greater anti-tumor efficacy.

Polymeric prodrug conjugate, 3, a 5-fluorouracil (5FU)-containing conjugate, employing a similar cathepsin B-cleavable peptide linking group (FIG. 1B). was studied against the murine colon cancer MC-38. implanted s.c. The equimolar dose of 25 mg 5FU/kg of the conjugate produced striking anti-tumor effects (70% tumor reduction, >6 days extension of time for tumors to reach 500 mg).

Plasmin labile construct, 26, bearing an aspartic acid-platinum-diamino-cyclohexane (DACH) chelate was studied using C57B1/6 mice bearing s.c. B16-F10 murine melanoma (FIG. 1C). (Single I.P. injection of construct on Day 7 following implantation of 1×10⁶ tumor cells. Tumor wt. =approx. 100 mg. Tumor reduction (% TR) calculated at time when median tumor in vehicle control mice reached the endpoint of 1, 500 mg, using the formula, 100×(1−T/C), where T=tumor volume in drug-treated mice and C=tumor volume in vehicle-treated mice. Tumor growth delay (T−C) calculated in days when tumors reached 1,000 mg. Construct, 26, reduced tumor size and delayed tumor growth in a dose-dependent fashion. The maximum tolerated dose (MTD) for 26 was 75 mg Pt/kg. This compared well with the MTD of oxaliplatin, a structurally related Pt drug, which was 10 mg Pt/kg. Thus, a substantially larger dose of Pt was safely delivered by the polymeric prodrug construct.

Conjugate, 31, (VEO-066) bearing the uPA-cleavable peptide -Pro-Gly-Arg-, and Pt chelated through an aspartic acid residue and DACH, reduced tumor size and delayed tumor growth in a dose-dependent manner (FIG. 1D).

The human colon cancer tumor HT-29 was implanted s.c. into athymic (nu/nu) mice. Construct, 6, (VEO-0003) the cathepsin-B-cleavable (-Gly-Phe-Leu-Gly-), Dox-containing conjugate was administered by daily i.v. injections for 5 days beginning when the tumors reached approx. 200 mg. It was possible to safely administer conjugate at twice the maximum tolerated dose of free Dox (6 mg Dox/kg) (FIG. 1E). The conjugate gave slightly better anti-tumor activity than the free drug.

All of the above-cited sources, patents, publications, and references are hereby expressly incorporated by way of reference in their respective entireties.

The invention being thus described, it will be obvious that same may be varied in many ways. Such variations are not to be regarded as a departure from the spirit and scope of the invention, and all such modifications are intended to be included within the scope of the following claims.

What is claimed is:

1. A polymeric drug conjugate comprising one or more biologically active agents conjugated via an enzymatically cleavable linker to:
(i) a regular repeating linear unit comprising a water soluble polymer segment and a multifunctional chemical moiety, or
(ii) a branched polymer comprising two or more water soluble polymer segments each bound to a common multifunctional chemical moiety.

2. The conjugate of claim 1, in which said one or more biologically active agents are conjugated via said linker to said multifunction chemical moiety of said regular repeating linear unit.

3. The conjugate of claim 1, in which said one or more biologically active agents are conjugated via said linker to at least one of said two or more water soluble polymer segments.

4. The conjugate of claim 1, in which said linker is cleaved by an intracellular enzyme.

5. The conjugate of claim 1, in which said linker is cleaved by an extracellular enzyme.

6. The conjugate of claim 1, in which said linker is cleaved by a membrane-bound enzyme.

7. The conjugate of claim 1, in which said linker is cleaved by an enzyme that is available at a target site.

8. The conjugate of claim 7, in which said enzyme is up-regulated at said target site.

9. The conjugate of claim 7, in which said target site is diseased tissue or biological fluid.

10. The conjugate of claim 9, in which said diseased tissue is present in skin, bone, cartilage, muscle, connective tissue, neural tissue, reproductive organs, endocrine tissue, lymphatic tissue, vasculature, or visceral organs.

11. The conjugate of claim 9, in which said biological fluid is blood, pleural fluid, peritoneal fluid, joint fluid, pancreatic fluid, bile, or cerebral-spinal fluid.

12. The conjugate of claim 1, in which the linker is cleaved by an enzyme resulting from a microbial infection, a skin surface enzyme, or an enzyme secreted by a cell.

13. The conjugate of claim 1, in which said linker is cleaved by an enzyme secreted by a cancer cell.

14. The conjugate of claim 1, in which said linker is cleaved by an enzyme located on the surface of a cancer cell.

15. The conjugate of claim 1, in which said linker is cleaved by an enzyme secreted by tissue associated with a chronic inflammatory disease.

16. The conjugate of claim 1, in which said linker is cleaved by an enzyme secreted by tissue associated with rheumatoid arthritis.

17. The conjugate of claim 1, in which said linker is cleaved by an enzyme secreted by tissue associated with osteoarthritis.

18. The conjugate of claim 1, in which said linker is further cleaved by hydrolysis, reduction reactions, oxidative reactions, pH shifts, photolysis, or combinations thereof.

19. The conjugate of claim 1, in which said linker is initially cleaved by hydrolysis, reduction reactions, oxidative reactions, pH shifts, photolysis, or combinations thereof and then further cleaved by an intracellular, extracellular, or membrane bound enzyme located at a target site.

20. The conjugate of claim 1, in which said linker is further cleaved by a non-specific enzyme reaction.

21. The conjugate of claim 2, in which said multifunctional chemical moiety is derived from a group selected from N-(2-hydroxyacetyl)serine, lysine, tris(2-aminoethyl)amine, N-(p-nitrophenylacetyl)-p-nitrophenylalanine acid hydrazide, 3,5-dihydroxyphenylacetic acid, 3,5-diaminobenzoic acid, 1,3-diamino-2-propanol, 2,2-diaminomethyl-1,3-dioxolane, and 6-amino-4-(2-aminoethyl)hexanoic acid.

22. The conjugate of claim 3, in which said common multifunctional chemical moiety comprises pentaerythritol, dendrimers, tris(2-aminoethyl)amine, or branched lysine trees.

23. The conjugate of claim 1, in which said water soluble polymer segment comprises a polymer with a molecular weight of about 400 to about 25,000.

24. The conjugate of claim 1, in which said water soluble polymer segment comprises poly(ethylene glycol), a copolymer of poly(ethylene glycol), or combinations thereof.

25. The conjugate of claim 1, in which said water soluble polymer segment comprises poly(vinyl alcohol), poly(2-hydroxyethyl methacrylate), poly(acrylic acid), poly(methacrylic acid), poly(maleic acid), poly(lysine), and the like, or combinations thereof.

26. The conjugate of claim 1, in which said linker comprises an amino acid, a sugar, a nucleic acid, or other organic compounds, or combinations thereof.

27. The conjugate of claim 1, in which said linker comprises a peptide sequence.

28. The conjugate of claim 1, in which said linker comprises a peptide sequence which can be cleaved by a serine protease.

29. The conjugate of claim 28, in which said serine protease is selected from the group consisting of thrombin, chymotrypsin, trypsin, elastase, kallikrein, and substilisin.

30. The conjugate of claim 29, in which said thrombin-cleavable peptide sequence comprises -Gly-Arg-Gly-Asp-, -Gly-Gly-Arg-, -Gly-Arg-Gly-Asp-Asn-Pro-, -Gly-Arg-Gly-Asp-Ser-, -Gly-Arg-Gly-Asp-Ser-Pro-Lys-, -Gly-Pro-Arg-, -Val-Pro-Arg-, or -Phe-Val-Arg-.

31. The conjugate of claim 29, in which said elastase-cleavable peptide sequence comprises -Ala-Ala-Ala-, -Ala-Ala-Pro-Val-, -Ala-Ala-Pro-Leu-, -Ala-Ala-Pro-Phe-, -Ala-Ala-Pro-Ala-, or -Ala-Tyr-Leu-Val-.

32. The conjugate of claim 1, in which said linker comprises a peptide sequence which can be cleaved by a cysteine proteinase.

33. The conjugate of claim 32, in which said cysteine proteinase is selected from the group consisting of papain, actinidin, bromelain, lysosomal cathepsins, cytosolic clpain, and parasitic protease.

34. The conjugate of claim 33, in which said parasitic protease is derived from Trypanosoma or Schistosoma.

35. The conjugate of claim 1, in which said linker comprises a peptide sequence which can be cleaved by an aspartic proteinase.

36. The conjugate of claim 35, in which said aspartic proteinase is selected from the group consisting of pepsin, chymosin, lysosomal cathepsins D, a processing enzyme, a fungal protease, and a viral proteinase.

37. The conjugate of claim 36, in which said processing enzyme comprises renin.

38. The conjugate of claim 36, in which said fungal protease comprises penicillopepsin, rhizopuspepsin, or endothiapepsin.

39. The conjugate of claim 36, in which said viral protease comprises the protease from te AIDS virus.

40. The conjugate of claim 1, in which said linker comprises a peptide sequence that can be cleaved by a matrix metalloproteinase.

41. The conjugate of claim 40, in which said matrix metalloproteinase is selected from the group consisting of collagenase, stromelysin, and gelatinase.

42. The conjugate of claim 40, in which said cleavable peptide sequence comprises -Gly-Pro-Asn-Gly-Ile-Ala-Gly-Asn-, -Gly-Pro-Gln-Gly-Ile-Ala-Gly-Asn-, -Gly-Pro-Asn-Gly-Ile-,Phe-Gly-Asn-, -Gly-Pro-Leu-Gly-Val-Arg-Gly-, -Gly-Pro-Leu-Gly-Met-Phe-Ala-Thr-, -Pro-Leu-Gly-Leu-Trp-Ala-, -Pro-Leu-Ala-Nva-Gly-Ala-, -Pro-Leu-Gly-Leu-Gly-Ala-, -Gly-Pro-Tyr-Ala-Pro-Ala-Gly-His-, -Gly-Pro-Asn-Gly-Ile-Leu-Gly-Asn-, -Pro-Leu-Gly-Met-Leu-Ser-, -Leu-Ile-Pro-Val-Ser-Leu-Ile-Ser-, -Gly-Pro-Leu-Gly-Pro-Z-, -Gly-Pro-Ile-Gly-Pro-Z-, -Pro-Leu-Gly-Pro-D-Arg-Z-, -Ala-Pro-Gly-Leu-Z-, -Pro-Leu-Gly-(Sleu)-Leu-Gly-Z-, -Pro-Gln-Gly-Ile-Ala-Gly-Trp-, -Pro-Leu-Gly-Cys(Me)-His-, -Pro-Leu-Gly-Leu-Trp-Ala-, -Pro-Leu-Ala-Leu-Trp-Ala-Arg-, -Pro-Leu-Ala-Tyr-Trp-Ala-Arg-, -Pro-Tyr-Ala-Tyr-Trp-Met-Arg-, -Pro-Leu-Gly-Met-Trp-Ser-Arg-, -Ala-Ala-Ala-, -Ala-Ala-Pro-Ala-, -Ala-Ala-Pro-Val-, -Ala-Ala-Pro-Leu-, -Ala-Ala-Pro-Phe-, -Ala-Tyr-Leu-Val-, -Gly-Pro-Y-Gly-Pro-Z-, -Gly-Pro-Leu-Gly-Pro-Z-, -Gly-Pro-Ile-Gly-Pro-Z-, -Leu-Gly-, Ile-Gly-, or -Ala-Pro-Gly-Leu-Z-, where Y and Z are amino acids.

43. The conjugate of claim 1, in which said linker comprises a peptide sequence that can be cleaved by an angiotensin converting enzyme.

44. The conjugate of claim 43, in which said angiotensin converting enzyme cleavable peptide sequence comprises -Asp-Lys-Pro-, -Gly-Asp-Lys-Pro-, or -Gly-Ser-Asp-Lys-Pro-.

45. The conjugate of claim 1, in which said linker comprises a peptide sequence that can be cleaved by a prostate specific antigen or a prostate specific membrane antigen.

46. The conjugate of claim 45, in which said linker includes -(Glu)ₙ-, and n is an integer from 1 to 10.

47. The conjugate of claim 1, in which said biologically active agent comprises an analgesic, an anesthetic, an antifungal, an antibiotic, an antiinflammatory, an anthelmintic, an antiarthritic, an antidote, an antiemetic, an antihistamine, an antihypertensive, an antimalarial, an antimicrobial, an antipsychotic, an antipyretic, an antiseptic, an antiarthritic, an antituberculotic, an antitussive, an antiviral, a cardioactive drug, a cathartic, a chemotherapeutic agent, a colored or fluorescent imaging agent, a corticoid, an antidepressant, a depressant, a diagnostic aid, a diuretic, an enzyme, an expectorant, a hormone, a hypnotic, a mineral, a nutritional supplement, a parasympathomimetic, a potassium supplement, a radiation sensitizer, a radioisotope; a receptor binding agent, a sedative, a sulfonamide, a stimulant, a sympathomimetic, a tranquilizer, a urinary antiinfective, a vasoconstrictor, a vasodilator, a vitamin, an xanthine derivative, or the like and combinations thereof.

48. The conjugate of claim 47, in which said chemotherapeutic agent comprises a nitrogen mustard, an ethylenimine, a methylmelamine, a nitrosourea, an alkyl sulfonate, a triazene, a folic acid analog, a pyrimidine analog, a purine analog, a vinca alkaloid, an epipodophyllotoxin, an antibiotic, an enzyme, a biological response modifier, a platinum complex, a methylhydrazine derivative, an adrenocorticol suppressant, a somatostatin, a somatostatin analog, a hormone, a hormone antagonist, or combinations thereof.

49. The conjugate of claim 48, in which said chemotherapeutic agent comprises methotrexate, taxol, aminopterin, doxorubicin, bleomycin, camptothecin, etoposide, estramustine, prednimustine, melphalan, hydroxyurea, or 5-fluorouracil.

50. The conjugate of claim 1, in which said biologically active agent comprises a peptide based pharmaceutical agent.

51. The conjugate of claim 50, in which said peptide based pharmaceutical agent comprises a cytokine, a growth factor, a cell receptor antagonist, or a cell receptor agonist.

52. The conjugate of claim 1, in which said biologically active agent comprises an eptifibatide and other platelet binding proteins, a granulocyte colony stimulating factor, a human growth factor, a vascular endothelial growth factor, a bone morphogenic protein, an interferon, or an interleukin.

53. The conjugate of claim 1, in which said biologically active agent comprises DNA, RNA, a DNA fragment, an RNA fragment, or a plasmid.

54. The conjugate of claim 2, comprising the structure: 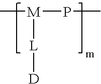
wherein P is said water soluble polymer segment, M is said multifunctional chemical moiety, L is said linker, D is said biologically active agent, and m is an integer.

55. The conjugate of claim 54, wherein m is an integer that is greater than or equal to 2.

56. The conjugate of claim 55, wherein m is an integer from about 2 to about 25.

57. The polymer conjugate of claim 55, in which said water-soluble polymer segment comprises poly(ethylene glycol) with a molecular weight of about 2,000, said multifunctional chemical moiety comprises L-lysine, said linker comprises (H-Gly-Phe-Gly-Gly-OEt), and said biologically active agent comprises 5-fluorouracil.

58. The conjugate of claim 55, in which said water-soluble polymer segment comprises poly(ethylene glycol) with a molecular weight of about 2,000, said multifunctional chemical moiety comprises L-lysine, said linker comprises (H-Gly-Phe-Leu-Gly-OH), and said biologically active agent comprises doxorubicin.

59. The conjugate of claim 55, in which said water-soluble polymer segment comprises poly(ethylene glycol) with a molecular weight of about 2,000, said multifunctional chemical moiety comprises 1,3-diamino-2-propanol, said linker comprises (H-Ser-Ser-Ser-Pro-Leu-Ala-Nva-Gly-Ala-OH), and said biologically active agent comprises an ethylenediamine chelated platinum dichloride salt.

60. The conjugate of claim 55, in which said water-soluble polymer comprises poly(ethylene glycol) with a molecular weight of about 2,000, said multifunctional chemical moiety comprises 1,3-diamino-2-propanol, said linker comprises (H-Ser-Ser-Ser-Gly-Pro-Asn-Gly-Ile-Ala-Gly-Asn-Asp-OH), and said biologically active substance comprises a 1,2-diaminocyclohexyl chelated platinum complex.

61. The conjugate of claim 3 comprising the structure:
Q(-P-L-D)ₖ
in which Q is said common multifunctional chemical moiety, P is said water soluble polymer segment, L is said linker, D is said biologically active agent, and k is an integer greater than or equal to 2.

62. The conjugate of claim 61, in which k is an integer from 2 to about 100.

63. A pharmaceutical composition comprising the conjugate of claim 1 and a physiologically acceptable carrier.

64. The pharmaceutical composition of claim 63, in which said composition is suitable for injection, or oral, topical, inhalation, or implantation methods of administration.

65. A method of alleviating a pathological condition comprising administering an effective amount of the conjugate of claim 1.

66. The method of claim 65, in which said pathological condition comprises neoplastic diseases, chronic inflammatory diseases acute inflammatory diseases, cardiac diseases, renal diseases, liver diseases, lung diseases, neurological diseases, musculoskeletal diseases, and immunological disorders.

67. The method of claim 65, comprising regulating cardiac function, renal function, liver function, lung function, or neurological function.

68. The method of claim 65, comprising modulating immunological function.

69. The method of claim 65, comprising modulating hormonal function.

70. The method of claim 65, comprising treating microbial infections.

71. The method of claim 65, comprising regulating scar tissue.

72. A method of targeting drug release comprising administering the conjugate of claim 1 and cleaving the linker with an enzyme that is available at a target site.

73. The method of claim 72, in which said target site is a site of disease.

74. A method of making the conjugate of claim 54, comprising:
(i) attaching said biologically active agent to said linker,
(ii) attaching said linker to said multifunctional chemical moiety, and
(iii) attaching said multifunctional chemical moiety to at least two of said water soluble polymer segments.

75. A method of making the conjugate of claim 54, comprising:
(i) attaching said biologically active agent to said linker,
(ii) attaching said multifunctional chemical moiety to at least two water soluble polymer segments, and
(iii) attaching said multifunctional chemical moiety to said linker.

76. A method of making the conjugate of claim 61, comprising:
(i) attaching said biologically active agent to said linker and attaching said linker to said water soluble polymer segment to form a construct, and
(ii) attaching at least two of said constructs to said common multifunctional chemical moiety via said water soluble polymer segment.

77. A method of making the conjugate of claim 61, comprising:
(i) attaching said biologically active agent to said linker,
(ii) attaching at least two of said water soluble polymer segments to said common multifunctional chemical moiety, and
(iii) attaching said linker to said water soluble polymer segments.
